(19) United States
(12) Patent Application Publication
(10) Pub. No.: US 2007/0135499 A1
(43) Pub. Date: Jun. 14, 2007

(54) Hydrazide compounds

(75) Inventors: Mitchell A. deLong, Raleigh, NC (US); Marcos L. Sznaidman, Durham, NC (US); Robert H. Oakley, Durham, NC (US); Allen E. Eckhardt, Durham, NC (US); Christine Hudson, Durham, NC (US); Jeffrey D. Yingling, Apex, NC (US); Michael Peel, Chapel Hill, NC (US); Thomas E. Richardson, Durham, NC (US); Clare Louise Murray, Chapel Hill, NC (US); Byappanahally N. Narasinga Rao, Chapel Hill, NC (US); Brian H. Heasley, Wake Forest, NC (US); Paresma R. Patel, San Diego, CA (US)

Correspondence Address: MICHAEL BEST & FRIEDRICH, LLP, 100 E WISCONSIN AVENUE, Suite 3300, MILWAUKEE, WI, 53202, US

(73) Assignee: Aerie Pharmaceuticals, Inc., Research Triangle Park, NC (US)

(21) Appl. No.: 11/485,182

(22) Filed: Jul. 11, 2006

Related U.S. Application Data
(60) Provisional application No. 60/698,190, filed on Jul. 11, 2005.

(51) Int. Cl. A61K 31/44

(52) U.S. Cl. 514/357; 546/332

(57) ABSTRACT

Hydrazide compounds with GPCR desensitization inhibitory activity are provided that may be used to influence, inhibit or reduce the action of a G-protein receptor kinase. Pharmaceutical compositions including therapeutically effective amounts of the hydrazide compounds and pharmaceutically acceptable carriers are also provided. Various methods using the compounds and/or compositions to affect disease states or conditions controlled or influenced by GPCRs are also provided. Various methods using the compounds and/or compositions to affect disease states or conditions such as cancer, osteoporosis and glaucoma are also provided.

### CROSS REFERENCE TO RELATED APPLICATIONS

This application claims the benefit of U.S. Provisional Patent Application No. 60/698,190, filed Jul. 11, 2005, which is incorporated herein by reference in its entirety.

### BACKGROUND

1. Field of the Invention

The present invention relates to hydrazide compounds that may affect the action of G protein-coupled receptor kinases in a cell and that are useful as therapeutic agents or with therapeutic agents. In particular, these compounds are useful in the treatment of eye diseases or ocular disorders such as glaucoma. The compounds can also be used to treat bone disease.

2. Background

A variety of hormones, neurotransmitters and biologically active substances control, regulate or adjust the functions of living bodies via specific receptors located in cell membranes. Many of these receptors mediate the transmission of intracellular signals by activating guanine nucleotide-binding proteins (G proteins) to which the receptor is coupled. Such receptors are generically referred to as G-protein coupled receptors (GPCRs) and include, among others, α-adrenergic receptors, β-adrenergic receptors, opioid receptors and prostaglandin receptors.

The G-protein coupled receptors play an important role in the regulation of various physiological functions. By way of example, GPCRs have been implicated in a number of disease states, including, but not limited to: cardiac indications such as angina pectoris, essential hypertension, myocardial infarction, supraventricular and ventricular arrhythmias, congestive heart failure, atherosclerosis, renal failure, diabetes, respiratory indications such as asthma, chronic bronchitis, bronchospasm, emphysema, airway obstruction, upper respiratory indications such as rhinitis, seasonal allergies, inflammatory disease, inflammation in response to injury, rheumatoid arthritis, chronic inflammatory bowel disease, glaucoma, hypergastrinemia, gastrointestinal indications such as acid/peptic disorder, erosive esophagitis, gastrointestinal hypersecretion, mastocytosis, gastrointestinal reflux, peptic ulcer, Zollinger-Ellison syndrome, pain, obesity, bulimia nervosa, depression, obsessive-compulsive disorder, organ malformations (for example, cardiac malformations), neurodegenerative diseases such as Parkinson's Disease and Alzheimer's Disease, multiple sclerosis, Epstein-Barr infection and cancer.

The balance between the initiation and the turn off of the intracellular signal, called desensitization, regulates the intensity and duration of the response of the receptors to stimuli such as agonists. Desensitization of agonist-occupied GPCRs is thought to result from their phosphorylation by specific kinases called G protein-coupled receptor kinases (GRKs) and the subsequent binding of arrestin proteins to the phosphorylated receptors. Arrestins are a family of intracellular proteins that bind activated GPCRs, including those that have been agonist-activated, and especially those that have been phosphorylated by G protein-coupled receptor kinases. The binding of the arrestins prevents further stimulation of G proteins and downstream signaling pathways. When desensitization occurs, the mediation or regulation of the physiological function mediated or regulated by the G proteins to which the receptors are coupled is reduced or prevented. For example, when agonists are administered to treat a disease or condition by activation of certain receptors, the receptors become desensitized from the action of the GRKs such that agonist administration may no longer result in therapeutic activation of the appropriate receptors. At that point, administration of the agonist no longer enables sufficient or effective control of or influence on the disease or condition intended to be treated.

In view of the role of GRKs in the desensitization of GPCRs, there is a need in the art for agents which prevent or reduce the desensitization of the GPCRs by controlling or inhibiting the action of the corresponding GRKs. Without wishing to be bound by theory, it is thought that certain compounds of the hydrazine class surprisingly exert their effects via inhibiting the activities of the GRKs.

### SUMMARY

A compound according to Formula (I) is provided: 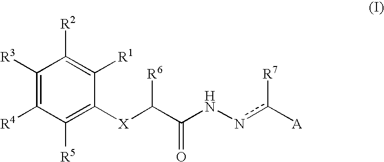
wherein [symbol]
may be a single or double bond;

A is a heteroaryl group (i): 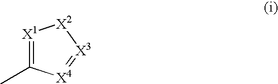
wherein X¹, X², X³ and X⁴ are, independently, CH, O, S or N—R⁶, with the proviso that at least one of X² or X³ is O, S or N—R⁶; or

A is a heteroaryl group (ii): 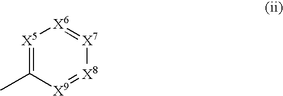
wherein X⁵and X⁹ are CH or C-halogen, X⁶ and X⁸ are CH, and X⁷ is N, and wherein the six-membered heteroaryl group may be further fused with an unsubstituted six-member aryl group;

R¹, R², R³, R⁴, and R⁵ are, independently, hydrogen; halogen; C₁-C₄ alkyl; amino; nitro; cyano; heteroaryl; carboxy; carbonylamino; aminosulfonyl; sulfonylamino; aminoacyl; thioalkyl; sulfonyl; acyl; heterocycle; —OR; —O—C₁-C₄alkyl-heterocycle; —C(O)NH—C₁-C₄alkyl-heterocycle; —C(O)NH-heteroaryl; —C(O)NH-aryl; or carboxylamino;

R is C₁-C₄ alkyl; aryl, heteroaryl, C₁-C₄ alkyl aryl or C₁-C₄ alkyl heteroaryl;

R⁶ is H or C₁-C₄ alkyl;

R⁷ is hydrogen, C₁-C₄ alkyl or C₁-C₄ alkoxy; and

X is O, S or N—R⁶.

A hydrazide compound according to Formula (II) is further provided:

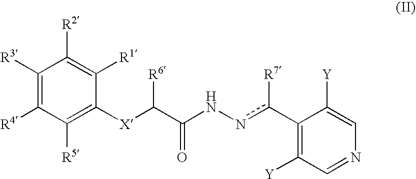

wherein

[symbol]

may be a single or double bond;

R^{1′}, R^{3′} and R^{5′} are hydrogen;

R^{2′} is hydrogen; halogen; C₁-C₄ alkyl; amino; nitro; cyano; heteroaryl; carbonylamino; aminosulfonyl; sulfonylamino; aminoacyl; thioalkyl; sulfonyl; acyl; heterocycle; —OR; —O—C₁-C₄alkyl-heterocycle; —C(O)NH—C₁-C₄alkyl-heterocycle; —C(O)NH-heteroaryl; —C(O)NH-aryl; or carboxylamino; with the proviso that when R^{2′} is not hydrogen, R^{4′} is hydrogen;

R^{4′} is hydrogen; halogen; C₁-C₄ alkyl; amino; nitro; cyano; heteroaryl; carbonylamino; aminosulfonyl; sulfonylamino; aminoacyl; thioalkyl; sulfonyl; acyl; heterocycle; —OR; —SR; —O—C₁-C₄alkyl-heterocycle; —C(O)NH—C₁-C₄alkyl-heterocycle;

—C(O)NH-heteroaryl; —C(O)NH-aryl; or carboxylamino; with the proviso that when R^{4′} is not hydrogen, R^{2′} is hydrogen;

R is C₁-C₄ alkyl; aryl, heteroaryl, C₁-C₄ alkyl aryl or C₁-C₄ alkyl heteroaryl;

R⁶ is H or C₁-C₄ alkyl;

R^{7′} is hydrogen, C₁-C₄ alkyl or C₁-C₄ alkoxy;

Y is halogen, and

X′ is O, S or N—R^{6′}.

In another embodiment, compositions comprising the compounds of Formula (I) and a pharmaceutically acceptable carrier, as well as compositions comprising the compounds of Formula (II) and a pharmaceutically acceptable carrier are provided.

In one embodiment, a pharmaceutical composition having GPCR desensitization inhibitory activity is provided for administration to a living organism, the pharmaceutical composition comprising a therapeutically effective amount of a compound according to Formula I or Formula II and a pharmaceutically acceptable carrier.

In a further embodiment, a method for influencing the action of a G-protein-coupled receptor kinase in a cell is provided comprising administering to or contacting with the cell at least one compound according to Formula (II). The method may be used to influence the action of a G-protein-coupled receptor kinase in a cell in vitro or in a cell in vivo in a living organism.

Another embodiment provides a method of reducing GPCR desensitization in a cell comprising administering to or contacting with the cell a therapeutically effective amount of a compound according to Formula (II). The method may be used reduce GPCR desensitization in a cell in vitro or in a cell in vivo in a living organism.

A further embodiment provides a method of inhibiting the action of a G-protein-coupled receptor kinase comprising applying to a medium or contacting with a cell an effective inhibitory amount of a compound according to Formula (II). The method may be used to inhibit the action of a GRK in a cell in vitro or in a cell in vivo in a living organism.

An additional embodiment provides a method of treating a condition comprising administering to a subject in need of treatment a safe and effective amount of a hydrazide derivative, wherein the condition is selected from the group consisting of eye disease, bone disorder (such as osteoporosis), heart disease, hepatic disease, renal disease, pancreatitis, cancer, myocardial infarct, gastric disturbance, hypertension, fertility control, nasal congestion, neurogenic bladder disorder, a gastrointestinal disorder, and a dermatological disorder. In one embodiment the condition comprises eye disease, and more particularly, glaucoma.

### BRIEF DESCRIPTION OF THE DRAWINGS

FIG. 1 is a graphical representation of the effect of a 60 minute pre-incubation of the GRK-2 inhibitor of Example 41 on ISO-induced β-arrestin translocation in β2 wt using a Transfluor® assay.

FIG. 2 is a graphical representation of the effect of a 60 minute pre-incubation of the GRK-2 inhibitor of Example 41 on ISO-induced β-arrestin translocation in β1 wt using a Transfluor® assay.

FIG. 3 is a graphical representation of the effect of a 60 minute pre-incubation of the GRK-2 inhibitor of Example 41 on morphine-induced β-arrestin translocation of mu-opioid receptor using a Transfluor® assay.

### DETAILED DESCRIPTION OF THE PREFERRED EMBODIMENTS

Novel hydrazide compounds and methods of using those compounds to reduce or prevent desensitization of GPCR pathways are provided.

“Alkyl” refers to a saturated aliphatic hydrocarbon including straight chain and branched chain groups. “Alkyl” may be exemplified by groups such as methyl, ethyl, n-propyl, isopropyl, n-butyl and the like. The alkyl group may be substituted or unsubstituted. When substituted, the substituent group is preferably amino, cyano, halogen, or hydroxyl. “C₁-C₄ alkyl” refers to alkyl groups with one to four carbon atoms.

“Acyl” refers to the group C(O)R wherein R includes C₁-C₄ alkyl, aryl, heteroaryl, C₁-C₄ alkyl aryl or C₁-C₄ alkyl heteroaryl.

“Alkoxy” refers to the group —O-alkyl wherein alkyl has the definition given above.

“Carboxyl” refers to the group —C(═O)O—,

“Carboxylamino” refers to the group —C(═O)O—NR′R′ where each R′ is, independently, hydrogen, C₁-C₄ alkyl, aryl, heteroaryl, C₁-C₄ alkyl aryl or C₁-C₄ alkyl heteroaryl.

“Carbonylamino” refers to the group —C(O)NR′R′ where each R′ is, independently, hydrogen, C₁-C₄ alkyl, aryl, heteroaryl, C₁-C₄ alkyl aryl or C₁-C₄ alkyl heteroaryl.

“Aminoacyl” refers to the group —NR′(CO)R′ wherein each R′ includes independently hydrogen or C₁-C₄ alkyl, aryl, heteroaryl, C₁-C₄ alkylaryl or C₁-C₄ alkyl heteroaryl.

“Aryl” refers to an aromatic carbocyclic group. “Aryl” may be exemplified by phenyl. The aryl group may be substituted or unsubstituted. When substituted, the substituent group is preferably C₁-C₄ alkyl, C₁-C₄ alkoxy, amino, cyano, halogen, or hydroxyl.

“C₁-C₄ alkyl aryl” refers to C₁-C₄ alkyl groups having an aryl substituent such that the aryl substituent is bonded through the alkyl group. “C₁-C₄ alkyl aryl” may be exemplified by benzyl.

“C₁-C₄ alkyl heteroaryl” refers to C₁-C₄ alkyl groups having a heteroaryl substituent such that the heteroaryl substituent is bonded through the alkyl group.

“Halogen” refers to fluoro, chloro, bromo or iodo atoms.

“Heteroaryl” refers to a monocyclic aromatic carbocyclic radical having one or more hetero atoms in the carbocyclic ring The heteroaryl group may be substituted or unsubstituted. When substituted, the substituents may be groups such as halogen, cyano, nitro or C₁-C₄ alkyl.

“Heterocycle” refers to a monocyclic saturated carbocyclic radical having one or two hetero atoms in the carbocyclic ring. Heterocycle may be exemplified by a morpholino radical.

“Thioalkyl” refers to the group —S-alkyl.

“Sulfonyl” refers to the —S(O)₂R′ group wherein R′ is hydrogen, C₁-C₄ alkyl, aryl, heteroaryl, C₁-C₄ alkyl aryl or C₁-C₄ alkyl heteroaryl.

“Sulfonylamino” refers to the —S(O)₂NH— group.

“Pharmaceutically acceptable carrier” means a carrier that is useful in preparing a pharmaceutical composition that is generally compatible with the other ingredients of the composition, not deleterious to the recipient, and neither biologically nor otherwise undesirable. “A pharmaceutically acceptable carrier” includes both one and more than one carrier. Embodiments include carriers for ocular, topical, parenteral, intravenous, intraperitoneal intramuscular, sublingual, nasal and oral administration. “Pharmaceutically acceptable carrier” also includes agents for preparation of aqueous dispersions and sterile powders for injection or dispersions.

“Excipient” as used herein includes physiologically compatible additives useful in preparation of a pharmaceutical composition. Examples of pharmaceutically acceptable carriers and excipients can for example be found in Remington Pharmaceutical Science, 16^{th} Ed.

“Therapeutically effective amount” as used herein refers to a dosage of the compounds or compositions effective for influencing, reducing, inhibiting or preventing desensitization of a receptor, particularly GPCR desensitization. This term as used herein may also refer to an amount effective at bringing about a desired in vivo effect in an animal, preferably, a human, such as a reduction in intraocular pressure.

“Administering” as used herein refers to administration of the compounds as needed to achieve the desired effect.

“Eye disease” as used herein includes, but is not limited to, glaucoma, allergy and dry eye.

The term “disease or condition associated with G-protein receptor kinase activity” is used to mean a disease or condition resulting, in whole or in part, from the effect on GPCR(s) by one or more GRKs.

The term “controlling the disease or condition” is used to mean changing the effect on GPCR(s) by one or more GRKs to affect the disease or condition.

“Desensitization” or “GPCR desensitization” refers generally to the process by which sensitized GPCRs are converted to desensitized GPCRs.

“Desensitized GPCR” means a GPCR that presently does not have the ability to respond to an agonist and activate conventional G protein signaling.

“Sensitized GPCR” means a GPCR that presently has ability to respond to agonist and activate conventional G protein signaling.

“GPCR desensitization pathway” means any cellular component of the GPCR desensitization process, as well as any cellular structure implicated in the GPCR desensitization process and subsequent processes, including but not limited to, arresting, GRKs, GPCRs, AP-2 protein, clathrin, protein phosphatases, and the like.

“GPCR signaling” means GPCR induced activation of G proteins. This may result in, for example, cAMP production.

“G protein-coupled receptor kinase” (GRK) includes any kinase that has the ability to phosphorylate a GPCR.

“GPCR desensitization inhibitory activity” of a composition (e.g., compound, solution, etc.) means that the composition is capable of inhibiting GPCR desensitization of at least one specific GPCR.

The term “to inhibit the G-protein receptor kinase activity” means to reduce or decrease the action of the GRK.

The term “to influence the GRK activity” or “to influence the action of the GRK” means to change or affect the action or activity of a GRK on one or more GPCRs.

The hydrazide compounds are represented by Formula I: 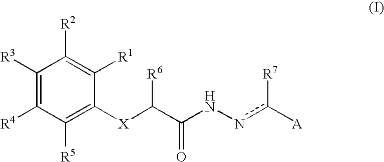
wherein [symbol]
may be a single or double bond;

A is heteroaryl group (i): 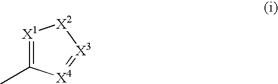
wherein X¹, X², X³ and X⁴ are, independently, CH, O, S or N—R⁶, with the proviso that at least one of X² or X³ is O, S or N—R⁶; or

A is heteroaryl group (ii): 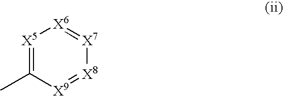
wherein X and X⁹ are CH or C-halogen, X⁶ and X⁸ are CH, and X⁷ is N, and wherein the six-membered heteroaryl group may be further fused with an unsubstituted six-member aryl group;

R¹, R², R³, R⁴, and R⁵ are, independently, hydrogen; halogen; C₁-C₄ alkyl; amino; nitro; cyano; heteroaryl; carbonylamino; aminosulfonyl; sulfonylamino; aminoacyl; thioalkyl; sulfonyl; acyl; heterocycle; carboxy; phenoxy; —OR; —SR; —O—C₁-C₄alkyl-heterocycle;

—C(O)NH-C₁-C₄alkyl-heterocycle; —C(O)NH-heteroaryl; —C(O)NH-aryl; or carboxylamino;

R is C₁-C₄ alkyl; aryl, heteroaryl, C₁-C₄ alkyl aryl or C₁-C₄ alkyl heteroaryl;

R⁶ is hydrogen or C₁-C₄ alkyl;

R⁷ is hydrogen, C₁-C₄ alkyl or C₁-C₄ alkoxy; and

X is O, S or N—R⁶.

In a preferred embodiment of Formula (I), X is NH. In a further preferred embodiment of Formula (I), each of R¹, R³ and R⁵ are hydrogen and at least one of R² or R⁴ is hydrogen. In another preferred embodiment, A is heteroaryl group (ii) and

[symbol]

is a double bond.

In another embodiment, the hydrazide compounds may be represented by formula (II): 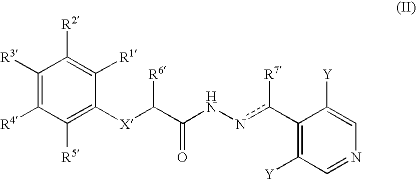
wherein [symbol]
indicates a single or double bond;

R^{1′}, R^{3′} and R^{5′} are hydrogen;

R^{2′} is hydrogen; halogen; C₁-C₄ alkyl; amino; nitro; cyano; heteroaryl; carbonylamino; aminosulfonyl; sulfonylamino; aminoacyl; thioalkyl; sulfonyl; acyl; heterocycle; carboxy; phenoxy; —OR; —SR; —O—C₁-C₄alkyl-heterocycle; —C(O)NH—C₁-C₄alkyl-heterocycle;

—(O)NH-heteroaryl; —C(O)NH-aryl; or carboxylamino; with the proviso that when R^{2′} is not hydrogen, R^{4′} is hydrogen; R^{4′} is hydrogen; halogen; C₁-C₄ alkyl; amino; nitro; cyano; heteroaryl; carbonylamino; aminosulfonyl; sulfonylamino; aminoacyl; thioalkyl; sulfonyl; acyl; heterocycle; —OR; —O—C₁-C₄alkyl-heterocycle; —C(O)NH—C₁-C₄alkyl-heterocycle; —C(O)NH-heteroaryl; —C(O)NH-aryl; or carboxylamino; with the proviso that when

R^{4′} is not hydrogen, R^{2′} is hydrogen;

R is C₁-C₄ alkyl; aryl, heteroaryl, C₁-C₄ alkyl aryl or C₁-C₄ alkyl heteroaryl;

R^{6′} is hydrogen or C₁-C₄ alkyl;

R^{7′} is hydrogen, C₁-C₄ alkyl or C₁-C₄ alkoxy;

Y is halogen, and

X′ is O, S or N—R^{6′}.

In some preferred embodiments of Formula (II), the hydrazides include those compounds wherein X′ is NH. In further preferred embodiments,

[symbol]

is a double bond. In other preferred embodiments, R^{6′} and R^{7′} are both hydrogen.

In one embodiment, the hydrazide compounds may be synthesized by the following general scheme:

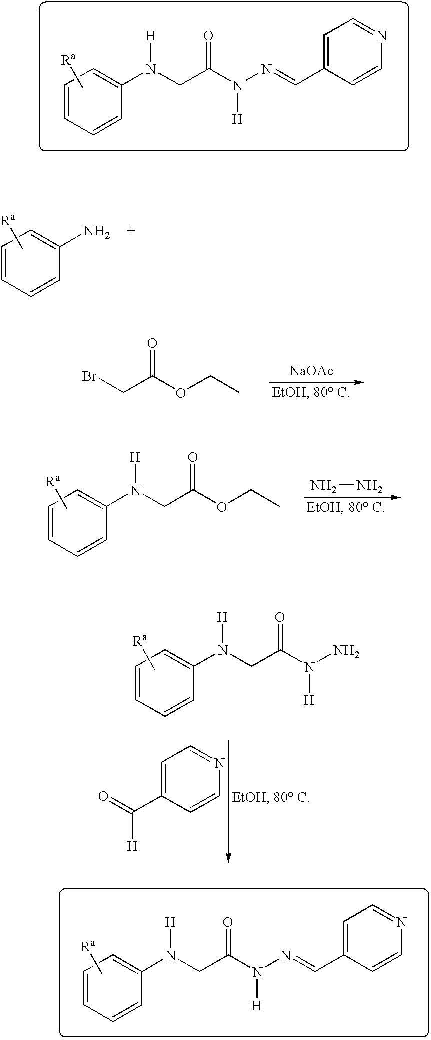

The R^{a} group generally represents the substituents as set forth in Formula (I) for groups R¹, R², R³, R⁴, and R⁵ and in Formula (II) for groups R^{1′}, R^{2′}, R^{3′}, R^{4′}, and R^{5′}. Other embodiments of the hydrazide derivatives may be synthesized by employing an alternate aldehyde or a ketone in the final step.

The hydrazide compounds of Formula (I) or Formula (II) and compositions including them typically have GPCR desensitization inhibitory activity and may be useful in influencing or inhibiting the action of G-protein receptor kinases, influencing, preventing or reducing the desensitization of receptors phosphorylated by G-protein receptor kinases, influencing or inhibiting other GRK-mediated events and in treatment and/or prevention of diseases or conditions controlled by receptors affected by one or more of the G-protein receptor kinases. The hydrazides may be used to influence or inhibit the action of GRKs either in a cell in vitro or in a cell in a living body in vivo. In one embodiment, a method is provided of inhibiting the action of a G-protein-coupled receptor kinase comprising applying to a medium such as an assay medium or contacting with a cell either in a cell in vitro or in a cell in a living body in vivo an effective inhibitory amount of a compound according to Formula (I) or (II). In a preferred embodiment, the GRK inhibited is GRK-2, GRK-3, GRK-5 or GRK-6. In a further preferred embodiment, the GRK inhibited is GRK-2.

In one embodiment, the hydrazides according to Formulas I or II are used in methods of reducing GPCR desensitization in a cell comprising administering to or contacting with the cell a therapeutically effective amount of one or more of the hydrazides. The one or more of the hydrazides are preferably administered in a pharmaceutically acceptable formulation, such as in or with a pharmaceutically acceptable carrier when the hydrazides are administered to a cell or cells in a living organism or body. In another embodiment, the hydrazides according to Formulas I or II are used in methods for influencing the action of a G-protein-coupled receptor kinase in a cell comprising administering to or contacting with the cell an effective amount of one or more hydrazides for influencing the action of the GRK in the cell. The one or more of the hydrazides are preferably administered in a pharmaceutically acceptable formulation, such as in or with a pharmaceutically acceptable carrier when the hydrazides are administered to a cell or cells in a living organism or body.

Treatment or prevention of diseases or conditions for which the hydrazides may be useful include any of the diseases or conditions associated with G-protein receptor kinase activity or diseases or conditions affected by GRK-mediated desensitization of GPCRs. By way of example, continuous exposure to endogenous stimuli can cause down-regulation and loss of response of beneficial GPCRs in certain hereditary as well as most chronic diseases. Examples of this type of disease behavior include the down-regulation and loss of response by both β-1 and β-2 adrenergic receptors in congestive heart failure. Desensitization via down-regulation of the receptors is also seen with exogenous administration of agonists or drugs such as morphine for pain or salbutamol for asthma, for example, in which desensitization of the receptors results in an undesired adverse effect known as drug tolerance. The hydrazides may be used to influence or reduce the GRK-controlled desensitization for conditions affected by the action or activity of GRKs, resulting in a therapeutic effect.

The hydrazides in some embodiments will be administered in conjunction with the administration of a therapeutic agent which is directed to influencing or controlling specific G-protein coupled receptors for the treatment or prevention of a condition or disease affected by those specific receptors. Combining administration of the hydrazides with a GPCR-directed therapeutic agent will provide a reduction or prevention of desensitization of the receptors to which the therapeutic agent is directed, resulting in improving the ability of the therapeutic agent to have the desired effect over a longer period of time. Additionally, the administration of the therapeutic agent or receptor agonist with an hydrazide formulation will enable lower doses of the therapeutic agent to be administered over a longer period of time.

One or more therapeutic agents may be administered with one or more hydrazide compounds. The therapeutic agents and/or the hydrazide compounds are preferably administered in a pharmaceutically acceptable formulation with a pharmaceutically acceptable carrier when the hydrazides are administered to a cell or cells in a living organism or body.

Compositions including the hydrazides of Formulas I or II may be obtained in the form of various salts or solvates. As the salts, physiologically acceptable salts or salts available as raw materials are used.

Pharmaceutical compositions for use with the hydrazides may be formulated in a conventional manner using one or more physiologically acceptable carriers or excipients. Thus, the compounds and their physiologically acceptable salts and solvates may be formulated for administration by, for example, eyedrop, in a topical oil-based formulation, injection, inhalation (either through the mouth or the nose), oral, buccal, parenteral or rectal administration. Techniques and formulations may generally be found in “Remington's Pharmaceutical Sciences”, (Meade Publishing Co., Easton, Pa.). Therapeutic compositions must typically be sterile and stable under the conditions of manufacture and storage.

Compositions of the present invention may comprise a safe and effective amount of the subject compounds, and a pharmaceutically-acceptable carrier. As used herein, “safe and effective amount” means an amount of a compound sufficient to significantly induce a positive modification in the condition to be treated, but low enough to avoid serious side effects (at a reasonable benefit/risk ratio), within the scope of sound medical judgment. A safe and effective amount of a compound will vary with the particular condition being treated, the age and physical condition of the patient being treated, the severity of the condition, the duration of the treatment, the nature of concurrent therapy, the particular pharmaceutically-acceptable carrier utilized, and like factors within the knowledge and expertise of the attending physician.

The route by which the compounds of the present invention (component A) will be administered and the form of the composition will dictate the type of carrier (component B) to be used. The composition may be in a variety of forms, suitable, for example, for systemic administration (e.g., oral, rectal, nasal, sublingual, buccal, or parenteral) or topical administration (e.g., local application on the skin, ocular, liposome delivery systems, or iontophoresis).

Carriers for systemic administration typically comprise at least one of a) diluents, b) lubricants, c) binders, d) disintegrants, e) colorants, f) flavors, g) sweeteners, h) antioxidants, j) preservatives, k) glidants, m) solvents, n) suspending agents, o) wetting agents, p) surfactants, combinations thereof, and others. All carriers are optional in the systemic compositions.

Ingredient a) is a diluent. Suitable diluents for solid dosage forms include sugars such as glucose, lactose, dextrose, and sucrose; diols such as propylene glycol; calcium carbonate; sodium carbonate; sugar alcohols, such as glycerin; mannitol; and sorbitol. The amount of ingredient a) in the systemic composition is typically about 50 to about 90%.

Ingredient b) is a lubricant. Suitable lubricants for solid dosage forms are exemplified by solid lubricants including silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of theobroma. The amount of ingredient b) in the systemic composition is typically about 5 to about 10%.

Ingredient c) is a binder. Suitable binders for solid dosage forms include polyvinylpyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The amount of ingredient c) in the systemic composition is typically about 5 to about 50%.

Ingredient d) is a disintegrant. Suitable disintegrants for solid dosage forms include agar, alginic acid and the sodium salt thereof, effervescent mixtures, croscarmelose, crospovidone, sodium carboxymethyl starch, sodium starch glycolate, clays, and ion exchange resins. The amount of ingredient d) in the systemic composition is typically about 0.1 to about 10%.

Ingredient e) for solid dosage forms is a colorant such as an FD&C dye. The amount of ingredient e) in the systemic composition is typically about 0.005 to about 0.1%.

Ingredient f) for solid dosage forms is a flavor such as menthol, peppermint, and fruit flavors. The amount of ingredient f) in the systemic composition is typically about 0.1 to about 1.0%.

Ingredient g) for solid dosage forms is a sweetener such as aspartame and saccharin. The amount of ingredient g) in the systemic composition is typically about 0.001 to about 1%.

Ingredient h) is an antioxidant such as butylated hydroxyanisole (“BHA”), butylated hydroxytoluene (“BHT”), and vitamin E. The amount of ingredient h) in the systemic composition is typically about 0.1 to about 5%.

Ingredient j) is a preservative such as benzalkonium chloride, methyl paraben and sodium benzoate. The amount of ingredient j) in the systemic composition is typically about 0.01 to about 5%.

Ingredient k) for solid dosage forms is a glidant such as silicon dioxide. The amount of ingredient k) in the systemic composition is typically about 1 to about 5%.

Ingredient m) is a solvent, such as water, isotonic saline, ethyl oleate, alcohols such as ethanol, and phosphate buffer solutions. The amount of ingredient m) in the systemic composition is typically from about 0 to about 100%.

Ingredient n) is a suspending agent. Suitable suspending agents include AVICEL® RC-591 (from FMC Corporation of Philadelphia, Pa.) and sodium alginate. The amount of ingredient n) in the systemic composition is typically about 1 to about 8%.

Ingredient o) is a surfactant such as lecithin, polysorbate 80, and sodium lauryl sulfate, and the TWEENS® from Atlas Powder Company of Wilmington, Del. Suitable surfactants include those disclosed in the C.T.F.A. Cosmetic Ingredient Handbook, 1992, pp. 587-592; Remington's Pharmaceutical Sciences, 15th Ed. 1975, pp. 335-337; and McCutcheon's Volume 1, Emulsifiers & Detergents, 1994, North American Edition, pp. 236-239. The amount of ingredient o) in the systemic composition is typically about 0.1% to about 2%.

Although the amounts of components A and B in the systemic compositions will vary depending on the type of systemic composition prepared, the specific derivative selected for component A and the ingredients of component B, in general, system compositions comprise 0.01% to 50% of component A and 50 to 99.99% of component B.

Compositions for parenteral administration typically comprise A) 0.1 to 10% of the compounds of the present invention and B) 90 to 99.9% of a carrier comprising a) a diluent and m) a solvent. In one embodiment, component a) comprises propylene glycol and m) comprises ethanol or ethyl oleate.

Compositions for oral administration can have various dosage forms. For example, solid forms include tablets, capsules, granules, and bulk powders. These oral dosage forms comprise a safe and effective amount, usually at least about 5%, and more particularly from about 25% to about 50% of component A). The oral dosage compositions further comprise about 50 to about 95% of component B), and more particularly, from about 50 to about 75%.

Tablets can be compressed, tablet triturates, enteric-coated, sugar-coated, film-coated, or multiple-compressed. Tablets typically comprise component A, and component B a carrier comprising ingredients selected from the group consisting of a) diluents, b) lubricants, c) binders, d) disintegrants, e) colorants, f) flavors, g) sweeteners, k) glidants, and combinations thereof. Specific diluents include calcium carbonate, sodium carbonate, mannitol, lactose and cellulose. Specific binders include starch, gelatin, and sucrose. Specific disintegrants include alginic acid and croscarmelose. Specific lubricants include magnesium stearate, stearic acid, and talc. Specific colorants are the FD&C dyes, which can be added for appearance. Chewable tablets preferably contain g) sweeteners such as aspartame and saccharin, or f) flavors such as menthol, peppermint, fruit flavors, or a combination thereof.

Capsules (including time release and sustained release formulations) typically comprise component A, and a carrier comprising one or more a) diluents disclosed above in a capsule comprising gelatin. Granules typically comprise component A, and preferably further comprise k) glidants such as silicon dioxide to improve flow characteristics.

The selection of ingredients in the carrier for oral compositions depends on secondary considerations like taste, cost, and shelf stability, which are not critical for the purposes of this invention. One skilled in the art would know how to select appropriate ingredients without undue experimentation.

The solid compositions may also be coated by conventional methods, typically with pH or time-dependent coatings, such that component A is released in the gastrointestinal tract in the vicinity of the desired application, or at various points and times to extend the desired action. The coatings typically comprise one or more components selected from the group consisting of cellulose acetate phthalate, polyvinyl acetate phthalate, hydroxypropyl methyl cellulose phthalate, ethyl cellulose, EUDRAGIT® coatings (available from Rohm & Haas G.M.B.H. of Darmstadt, Germany), waxes and shellac.

Compositions for oral administration can also have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically comprise component A and component B, namely, a carrier comprising ingredients selected from the group consisting of a) diluents, e) colorants, f) flavors, g) sweeteners, j) preservatives, m) solvents, n) suspending agents, and o) surfactants. Peroral liquid compositions preferably comprise one or more ingredients selected from the group consisting of e) colorants, f) flavors, and g) sweeteners.

Other compositions useful for attaining systemic delivery of the subject compounds include sublingual, buccal and nasal dosage forms. Such compositions typically comprise one or more of soluble filler substances such as a) diluents including sucrose, sorbitol and mannitol; and c) binders such as acacia, microcrystalline cellulose, carboxymethyl cellulose, and hydroxypropyl methylcellulose. Such compositions may further comprise b) lubricants, e) colorants, f) flavors, g) sweeteners, h) antioxidants, and k) glidants.

In one embodiment of the invention, the compounds of the present invention are topically administered. Topical compositions that can be applied locally to the eye may be in any form known in the art, non-limiting examples of which include gelable drops, spray, ointment, or a sustained or non-sustained release unit placed in the conjunctival cul-du-sac of the eye.

Topical compositions that can be applied locally to the skin may be in any form including solutions, oils, creams, ointments, gels, lotions, shampoos, leave-on and rinse-out hair conditioners, milks, cleansers, moisturizers, sprays, skin patches, and the like. Topical compositions comprise: component A, the compounds described above, and component B, a carrier. The carrier of the topical composition preferably aids penetration of the compounds into the eye. Component B may further comprise one or more optional components.

The dosage range of the compound for systemic administration is from about 0.01 to about 1000 μg/kg body weight, preferably from about 0.1 to about 100 μg/kg per body weight, most preferably form about 1 to about 50 μg/kg body weight per day. The transdermal dosages will be designed to attain similar serum or plasma levels, based upon techniques known to those skilled in the art of pharmacokinetics and transdermal formulations. Plasma levels for systemic administration are expected to be in the range of 0.01 to 100 nanograms/ml, more preferably from 0.05 to 50 ng/ml, and most preferably from 0.1 to 10 ng/ml. While these dosages are based upon a daily administration rate, weekly or monthly accumulated dosages may also be used to calculate the clinical requirements.

Dosages may be varied based on the patient being treated, the condition being treated, the severity of the condition being treated, the route of administration, etc. to achieve the desired effect.

The compounds of the present invention are also useful in decreasing intraocular pressure. Thus, these compounds are useful in the treatment of glaucoma. The preferred route of administration for treating glaucoma is topically.

The exact amounts of each component in the topical composition depend on various factors. The amount of component A added to the topical composition is dependent on the IC₅₀ of component A, typically expressed in nanomolar (nM) units. For example, if the IC₅₀ of the medicament is 1 nM, the amount of component A will be from about 0.0001 to about 0.01%. If the IC₅₀ of the medicament is 10 nM, the amount of component A) will be from about 0.001 to about 0.1%. If the IC₅₀ of the medicament is 100 nM, the amount of component A will be from about 0.01 to about 1.0%. If the IC₅₀ of the medicament is 1000 nM, the amount of component A will be 0.1 to 10%, preferably 0.5 to 5.0%. If the amount of component A is outside the ranges specified above (i.e., either higher or lower), efficacy of the treatment may be reduced. IC₅₀ can be calculated according to the method in Reference Example 1, below. One skilled in the art would know how to calculate an IC₅₀. The remainder of the composition, up to 100%, is component B.

The amount of the carrier employed in conjunction with component A is sufficient to provide a practical quantity of composition for administration per unit dose of the medicament. Techniques and compositions for making dosage forms useful in the methods of this invention are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2^{nd} Ed., (1976).

Component B may comprise a single ingredient or a combination of two or more ingredients. In the topical compositions, component B comprises a topical carrier. Suitable topical carriers comprise one or more ingredients selected from the group consisting of phosphate buffered saline, isotonic water, deionized water, monofunctional alcohols, symmetrical alcohols, aloe vera gel, allantoin, glycerin, vitamin A and E oils, mineral oil, propylene glycol, PPG-2 myristyl propionate, dimethyl isosorbide, castor oil, combinations thereof, and the like. More particularly, carriers for skin applications include propylene glycol, dimethyl isosorbide, and water, and even more particularly, phosphate buffered saline, isotonic water, deionized water, monofunctional alcohols and symmetrical alcohols.

The carrier of the topical composition may further comprise one or more ingredients selected from the group consisting of q) emollients, r) propellants, s) solvents, t) humectants, u) thickeners, v) powders, w) fragrances, x) pigments, and y) preservatives.

Ingredient q) is an emollient. The amount of ingredient q) in a skin-based topical composition is typically about 5 to about 95%. Suitable emollients include stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate, and combinations thereof. Specific emollients for skin include stearyl alcohol and polydimethylsiloxane.

Ingredient r) is a propellant. The amount of ingredient r) in the topical composition is typically about 0 to about 95%. Suitable propellants include propane, butane, isobutane, dimethyl ether, carbon dioxide, nitrous oxide, and combinations thereof.

Ingredient s) is a solvent. The amount of ingredient s) in the topical composition is typically about 0 to about 95%. Suitable solvents include water, ethyl alcohol, methylene chloride, isopropanol, castor oil, ethylene glycol monoethyl ether, diethylene glycol monobutyl ether, diethylene glycol monoethyl ether, dimethylsulfoxide, dimethyl formamide, tetrahydrofuran, and combinations thereof. Specific solvents include ethyl alcohol and homotopic alcohols.

Ingredient t) is a humectant. The amount of ingredient t) in the topical composition is typically 0 to 95%. Suitable humectants include glycerin, sorbitol, sodium 2-pyrrolidone-5-carboxylate, soluble collagen, dibutyl phthalate, gelatin, and combinations thereof. Specific humectants include glycerin.

Ingredient u) is a thickener. The amount of ingredient u) in the topical composition is typically about 0 to about 95%.

Ingredient v) is a powder. The amount of ingredient v) in the topical composition is typically 0 to 95%. Suitable powders include beta-cyclodextrins, hydroxypropyl cyclodextrins, chalk, talc, fullers earth, kaolin, starch, gums, colloidal silicon dioxide, sodium polyacrylate, tetra alkyl ammonium smectites, trialkyl aryl ammonium smectites, chemically-modified magnesium aluminum silicate, organically-modified montmorillonite clay, hydrated aluminum silicate, fumed silica, carboxyvinyl polymer, sodium carboxymethyl cellulose, ethylene glycol monostearate, and combinations thereof. For ocular applications, specific powders include beta-cyclodextrin, hydroxypropyl cyclodextrin, and sodium polyacrylate. For gel dosing ocular formulations, sodium polyacrylate may be used.

Ingredient w) is a fragrance. The amount of ingredient w) in the topical composition is typically about 0 to about 0.5%, particularly, about 0.001 to about 0.1%. For ocular applications a fragrance is not generally used.

Ingredient x) is a pigment. Suitable pigments for skin applications include inorganic pigments, organic lake pigments, pearlescent pigments, and mixtures thereof. Inorganic pigments useful in this invention include those selected from the group consisting of rutile or anatase titanium dioxide, coded in the Color Index under the reference CI 77,891; black, yellow, red and brown iron oxides, coded under references CI 77,499, 77,492 and, 77,491; manganese violet (CI 77,742); ultramarine blue (CI 77,007); chromium oxide (CI 77,288); chromium hydrate (CI 77,289); and ferric blue (CI 77,510) and mixtures thereof.

The organic pigments and lakes useful in this invention include those selected from the group consisting of D&C Red No. 19 (CI 45,170), D&C Red No. 9 (CI 15,585), D&C Red No. 21 (CI 45,380), D&C Orange No. 4 (CI 15,510), D&C Orange No. 5 (CI 45,370), D&C Red No. 27 (CI 45,410), D&C Red No. 13 (CI 15,630), D&C Red No. 7 (CI 15,850), D&C Red No. 6 (CI 15,850), D&C Yellow No. 5 (CI 19,140), D&C Red No. 36 (CI 12,085), D&C Orange No. 10 (CI 45,425), D&C Yellow No. 6 (CI 15,985), D&C Red No. 30 (CI 73,360), D&C Red No. 3 (CI 45,430), the dye or lakes based on Cochineal Carmine (CI 75,570) and mixtures thereof.

The pearlescent pigments useful in this invention include those selected from the group consisting of the white pearlescent pigments such as mica coated with titanium oxide, bismuth oxychloride, colored pearlescent pigments such as titanium mica with iron oxides, titanium mica with ferric blue, chromium oxide and the like, titanium mica with an organic pigment of the above-mentioned type as well as those based on bismuth oxychloride and mixtures thereof. The amount of pigment in the topical composition is typically about 0 to about 10%. For ocular applications a pigment is not generally used.

In a particularly preferred embodiment of the invention, topical pharmaceutical compositions for ocular administration are prepared typically comprising component A and B (a carrier), such as purified water, and one or more ingredients selected from the group consisting of y) sugars or sugar alcohols such as dextrans, particularly dextran 70, z) cellulose or a derivative thereof, aa) a salt, bb) disodium EDTA (Edetate disodium), and cc) a pH adjusting additive.

Examples of z) cellulose derivatives suitable for use in the topical pharmaceutical composition for ocular administration include sodium carboxymethylcellulose, ethylcellulose, methylcellulose, and hydroxypropyl-methylcellulose, particularly, hydroxypropyl-methylcellulose.

Examples of aa) salts suitable for use in the topical pharmaceutical composition for ocular administration include mono-, di- and trisodium phosphate, sodium chloride, potassium chloride, and combinations thereof.

Examples of cc) pH adjusting additives include HCl or NaOH in amounts sufficient to adjust the pH of the topical pharmaceutical composition for ocular administration to 6.8-7.5.

Component A may be included in kits comprising component A, a systemic or topical composition described above, or both; and information, instructions, or both that use of the kit will provide treatment for cosmetic and medical conditions in mammals (particularly humans). The information and instructions may be in the form of words, pictures, or both, and the like. In addition or in the alternative, the kit may comprise the medicament, a composition, or both; and information, instructions, or both, regarding methods of application of medicament, or of composition, preferably with the benefit of treating or preventing cosmetic and medical conditions in mammals (e.g., humans).

The invention will be further explained by the following illustrative examples that are intended to be non-limiting.

### EXAMPLES

Procedures for preparation of the hydrazides are described in the following examples. All temperatures are given in degrees Centigrade. Reagents were purchased from commercial sources or prepared following literature procedures. Unless otherwise noted, HPLC purification was performed by redissolving the residue in a small volume of DMSO and filtering through a 0.45 micron (nylon disc) syringe filter. The solution was then purified using a 50 mm Varian Dynamax HPLC 21.4 mm Microsorb Guard-8 C₈ column. The initial concentration of 40-80% MeOH:H₂O was selected as appropriate for the target compound. This initial gradient was maintained for 0.5 minutes then increased to 100% MeOH:0% H₂O over 5 minutes. 100% MeOH was maintained for 2 more minutes before it was re-equilibrated back to the initial starting gradient. Total run time was 8 minutes. The resulting fractions were analyzed, combined as appropriate, and then evaporated to provide purified material.

Proton magnetic resonance (¹H NMR) spectra were recorded on either a Varian INOVA 400 MHz (¹H) NMR spectrometer, Varian INOVA 500 MHz (¹H) NMR spectrometer, Bruker ARX 300 MHz (¹H) NMR spectrometer, Bruker DPX 400 MHz (¹H) NMR spectrometer, or a Bruker DRX 500 MHz (¹H) NMR spectrometer. All spectra were determined in the solvents indicated. Although chemical shifts are reported in ppm downfield of tetramethylsilane, they are referenced to the residual proton peak of the respective solvent peak for ¹H NMR. Interproton coupling constants are reported in Hertz (Hz). Analytical HPLC was performed using a Phenomenex Aqua 5 micron C₁₈ 125 Å 50×4.60 mm column coupled with an Agilent 1100 series VWD UV detector. A neutral 0.1% BES (w/v) pH 7.1 buffer with LiOH and 1% CH₃CN in H₂O is used as the aqueous phase. The initial gradient was 55% MeOH aqueous buffer which was increased to 100% MeOH over 3 minutes. 100% MeOH was maintained for 2 minutes before it was re-equilibrated to the initial starting gradient. Spectra were analyzed at 254 nm. LCMS spectra were obtained using a Thermofinnigan AQA MS ESI instrument. The samples were passed through a Phenomenex Aqua 5 micron C₁₈ 125 Å 50×4.60 mm column. The initial gradient was 55% MeOH: 1% CH₃CN in H₂O which was increased to 100% MeOH over 3 minutes. 100% MeOH was maintained for 2 minutes before it was re-equilibrated to the initial starting gradient. The spray setting for the MS probe was at 350 μL/min with a cone voltage at 25 mV and a probe temperature at 450 ° C.

The following Examples 1-101 illustrate procedures for the preparation of intermediates and methods for the preparation of hydrazides.

### Reference Example One

The inhibition of G-protein-coupled receptor kinases including hGRK-2 was determined for the hydrazide compounds using a biochemical assay. The results for hGRK-2 are given in each example below. The inhibition of GRK-3, GRK-5 and GRK-6 was also determined using the same assay for some of the hydrazide derivatives. These results are given for the hydrazide compounds tested according to the following scale provided in terms of Ki (nM):

| | | |
|---|---|---|
| 10-100 | nM Ki | ++++ |
| 100-1000 | nM Ki | +++ |
| 1000-10,000 | nM Ki | ++ |
| >10,000 | nM Ki | +. |

The protein kinase inhibition was determined using a biochemical assay utilizing the light emission of a luciferase reaction. The luciferase-based assay operates on the following reaction principles:

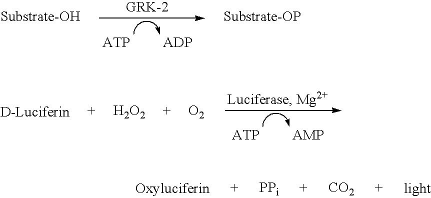

An inhibitor of GRK-2 will increase in the amount of ATP in solution, as shown. Thus, an inhibitor of GRK-2 will drive the luciferase reaction to the right, resulting in more light emitted. The amount of light emitted is proportional to the inhibition resulting from the GRK-2 inhibitor. The luciferase-based assay was also used to test some of the hydrazides for the inhibition properties towards other kinases.

The test procedure was as follows:

Assay Buffer:

50 mM HEPES, pH 7.5
10 mM MgCl₂
100 μM activated sodium orthovanadate
0.01% CHAPS
0.1% BSA
1 mM DTT (added fresh daily)
   10× Assay Buffer Stock:
500 mM HEPES, pH 7.5
100 mM MgCl₂
1 mM activated sodium orthovanadate
0.1% CHAPS
1% BSA (leave out for 10× compound dilution buffer)
   Final Assay Conditions:
50 μM test compound
20 μM casein
10 μM ATP
50 nM hGRK2
4.5% DMSO
90-120 minute incubation

Stopped by addition of 30 μl 3×-diluted Kinase-Glo reagent containing 0.01% trypan blue. Counted on FUSION plate reader.

Protocol

Compound Dilution and Transfer

Prepare sufficient buffer containing 40% DMSO to add 20 μl/well to the number of plates being assayed. This buffer should not contain BSA because it will precipitate with addition of 40% DMSO. (EXAMPLE: To make 3000 ml compound dilution buffer: 1200 ml DMSO, 180 ml 10× compound dilution buffer, 1615 ml ultrapure water, 3 ml 1M DTT.).

Using Multidrop, add 20 μl of compound dilution buffer to all wells of the 384-well daughter plate (add 1 μl DMSO to control wells). This will result in a daughter plate which contains 21 μl of ˜500 μM test compound.

Using the PlateTrak, transfer 5 μl of the test compounds to a 384-well, non-binding, white microtiter plate.

GRK2/ATP Addition

Prepare sufficient volume of buffer (with BSA and 1 mM DTT) containing 125 nM GRK2 and 25 μM ATP. (Example: To 219 ml of buffer was added 550 μl 10 mM ATP and 350 μl 79 μM GRK2).

Using Multidrop, add 20 μl of GRK2/ATP mixture to all wells of the microtiter plate.

Casein Addition

Prepare sufficient volume of buffer (with BSA and 1 mM DTT) containing 40 μM casein. (EXAMPLE: To 211 ml buffer add 8.8 ml 1 mM casein)

Using Multidrop, add 25 μl to columns 1 through 23 of the microtiter plate. Add 25 μl of complete buffer to column 24 (blanks).

Incubation

Mix reaction gently by tapping (Multidrop addition of casein does a decent job of mixing), stack plates and incubate at room temperature for between 90 and 120 minutes. Assay progression can be tracked in a separate plate if desired. Target 20-30% ATP consumption. Try to avoid exceeding 40% consumption as kinetics might become non-linear due to substrate (ATP) depletion.

Addition of Kinase-Glo Reagents

The Kinase-Glo reagent can be diluted 3-fold with no loss of data quality in this assay. Additionally, since the library contains many colored compounds which will quench the light emitted from the well resulting in a false negative (kinase inhibitors result in less ATP consumption thus more light emitted), the entire reaction is subject to intentional quench to override this effect. This intentional quench is accomplished by the addition of 0.01% trypan blue to the Kinase-Glo reagents. (EXAMPLE: 100 ml 1× Kinase-Glo reagents (prepared according to product insert), 200 ml assay buffer, 7.5 ml 0.4% trypan blue.

Using Multidrop, add 30 μl to all wells of the assay plate.

Count on FUSION plate reader in luminescence mode.

### Example 1

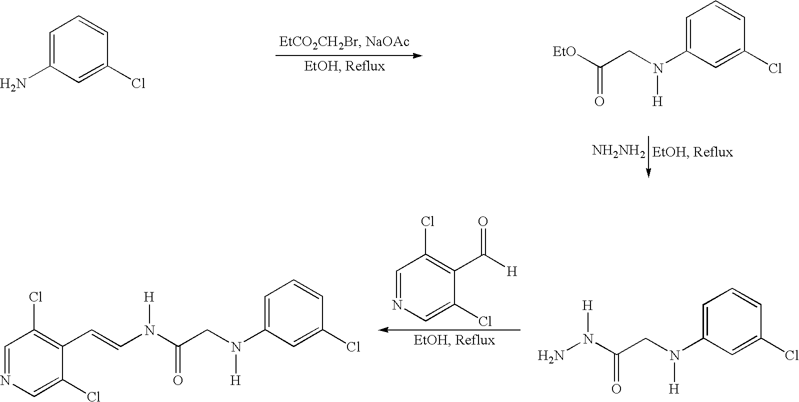

Step 1: (3-Chloro-phenylamino)-acetic acid ethyl ester: A 250-mL round bottom flask equipped with a stirrer bar was charged with 3-chloroaniline (10 mL, 94.5 mmol), ethanol (50 mL), sodium acetate (15.5 g, 189 mmol), and bromoethylacetate (10.4 mL, 94.5 mmol) and heated to reflux for 4 h. The reaction was allowed to cool to room temperature and 50 mL of H₂O was added to afford a precipitate. The product was collected by filtration and recrystallized from ethanol to afford (3-Chloro-phenylamino)-acetic acid ethyl ester (10.1 g, 50%) as a solid. ¹H NMR (400 MHz, CHLOROFORM-d) δ ppm 1.27-1.34 (m, 3H) 3.87 (s, 2H) 4.25 (q, J=7.16 Hz, 2H) 4.37 (s, 1H) 6.44-6.51 (m, 1H) 6.57 (t, J=2.15 Hz, 1H) 6.68-6.73 (m, 1H) 7.08 (t, J=8.00 Hz, 1H); LCMS: 214 (M+H).

Step 2: (3-Chloro-phenylamino)-acetic acid hydrazide: A 100-mL round bottom flask equipped with a stirrer bar was charged with (3-Chloro-phenylamino)-acetic acid ethyl ester (5.0 g, 23.4 mmol), ethanol (28 mL), and hydrazine hydrate (3.4 mL mL, 10 eq) and heated to reflux for 48 h. The reaction was allowed to cool to room temperature and concentrated in vacuo. Water was added to the flask to afford a precipitate. The precipitate was collected by filtration to afford (3-Chloro-phenylamino)-acetic acid hydrazide (4.7 g, 98%) as a solid. ¹H NMR (400 MHz, CHLOROFORM-d) δ ppm 3.80-3.94 (m, J=5.66 Hz, 4 H) 4.28 (s, 1 H) 6.42-6.50 (m, 1 H) 6.58 (t, J=2.15 Hz, 1 H) 6.73-6.82 (m, 1 H) 7.11 (t, J=8.00 Hz, 1 H) 7.62 (s, 1 H); LCMS: 200 (M+H).

Step 3: (3-Chloro-phenylamino)-acetic acid [1-(3,5-dichloro-pyridin-4-yl)-meth-(E)-ylidene]-hydrazide: A 5.0 mL vial equipped with a stirrer bar was charged with (3-Chloro-phenylamino)-acetic acid hydrazide (75 mg, 0.38 mmol), 3,5-dichloro-4-pyridinecarboxaldehyde (67 mg, 0.38 mmol) in EtOH (1.0 mL) and heated to 90° C. for 2 h then cooled to room temperature. The resulting precipitate was collected by filtration to afford (3-Chloro-phenylamino)-acetic acid [1-(3,5-dichloro-pyridin-4-yl)-meth-(E)-ylidene]-hydrazide (89 mg, 67%). ¹H NMR (400 MHz, DMSO-d₆) δ ppm 4.21 (d, J=6.05 Hz, 2H) 6.47-6.60 (m, 3H) 7.05 (t, J=8.00 Hz, 1H) 8.25 (s, 1H) 8.69 (s, 1H) 11.96 (s, 1H); LCMS: 357 (M+H).

Results of the biochemical assay described above:

| | |
|---|---|
| GRK-2 | ++ |
| GRK-3 | ++ |
| GRK-5 | ++ |
| GRK-6 | −−. |

### Example 2

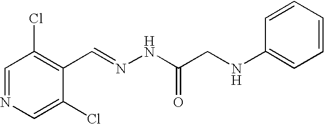

Phenylamino-acetic acid [1-(3,5-dichloro-pyridin-4-yl)-meth-(E)-ylidene]-hydrazide: In a similar fashion by making the appropriate substitution for the aniline, the following compound was prepared according to Example 1: ¹H NMR (400 MHz, DMSO-d₆) δ ppm 4.19 (d, J=6.05 Hz, 2H) 5.74 (t, J=5.95 Hz, 1H) 6.49-6.60 (m, 4H) 7.05 (t, J=7.81 Hz, 2H) 8.26 (s, 1H) 8.69 (s, 1H) 11.95 (s, 1H); LCMS: 323 (M+H).

Results of the biochemical assay described above: GRK-2 +++.

### Example 3

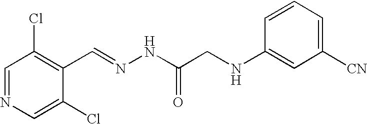

(3-Cyano-phenylamino)-acetic acid [1-(3,5-dichloro-pyridin-4-yl)-meth-(E)-ylidene]-hydrazide: In a similar fashion by making the appropriate substitution for the aniline, the following compound was prepared according to Example 1: ¹H NMR (400 MHz, DMSO-d₆) δ ppm 4.25 (d, J=6.05 Hz, 2H) 6.35-6.41 (m, 1H) 6.86-6.98 (m, 3H) 7.23 (dd, J=9.08, 7.71 Hz, 1H) 8.26 (s, 1H) 8.69 (s, 1H) 11.98 (s, 1H); LCMS: 348 (M+H).

Results of the biochemical assay described above: GRK-2 ++.

### Example 4

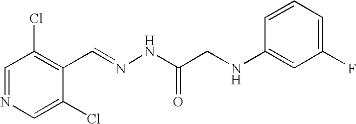

(3-Fluoro-phenylamino)-acetic acid [1-(3,5-dichloro-pyridin-4-yl)-meth-(E)-ylidene]-hydrazide: In a similar fashion by making the appropriate substitution for the aniline, the following compound was prepared according to Example 1: ¹H NMR (400 MHz, DMSO-d₆) δ ppm 4.20 (d, J=6.05 Hz, 2H) 6.12-6.18 (m, 1H) 6.25-6.43 (m, 4H) 7.05 (q, J=7.87 Hz, 1H) 8.26 (s, 1H) 8.69 (s, 1H) 11.97 (s, 1H); LCMS: 341 (M+H).

Results of the biochemical assay described above:

| | |
|---|---|
| GRK-2 | +++ |
| GRK-3 | ++ |
| GRK-5 | ++ |
| GRK-6 | −−. |

### Example 5

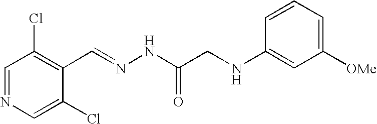

(3-Methoxy-phenylamino)-acetic acid [1-(3,5-dichloro-pyridin-4-yl)-meth-(E)-ylidene]-hydrazide: In a similar fashion by making the appropriate substitution for the aniline, the following compound was prepared according to Example 1: ¹H NMR (400 MHz, DMSO-d₆) δ ppm 3.63 (s, 3H) 4.17 (d, J=6.05 Hz, 2H) 5.75-5.80 (m, 1H) 6.09-6.19 (m, 4H) 6.94 (t, J=7.91 Hz, 1H) 8.26 (s, 1H) 8.69 (s, 1 H) 11.95 (s, 1H); LCMS: 353 (M+H).

Results of the biochemical assay described above:

| | |
|---|---|
| GRK-2 | ++++ |
| GRK-3 | ++++ |
| GRK-5 | +++ |
| GRK-6 | +. |

### Example 6

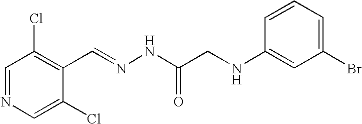

(3-Bromo-phenylamino)-acetic acid [1-(3,5-dichloro-pyridin-4-yl)-meth-(E)-ylidene]-hydrazide: In a similar fashion by making the appropriate substitution for the aniline, the following compound was prepared according to Example 1: ¹H NMR (400 MHz, DMSO-d₆) δ ppm 4.20 (d, J=6.05 Hz, 2H) 6.16 (t, J=5.95 Hz, 1H) 6.52-6.56 (m, 1H) 6.66 (d, J=8.79 Hz, 1H) 6.70-6.73 (m, J=1.76 Hz, 1H) 6.99 (t, J=7.91 Hz, 1H) 8.25 (s, 1H) 8.69 (s, 1H) 11.96 (s, 1H); LCMS: 401 (M+H).

Results of the biochemical assay described above: GRK-2 ++.

### Example 7

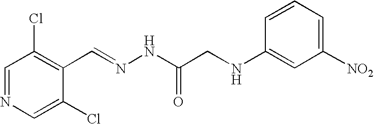

(3-Nitro-phenylamino)-acetic acid [1-(3,5-dichloro-pyridin-4-yl)-meth-(E)-ylidene]-hydrazide: In a similar fashion by making the appropriate substitution for the aniline the following compound was prepared according to Example 1: ¹H NMR (400 MHz, DMSO-d₆) δ ppm 4.31 (d, J=5.86 Hz, 2H) 6.63 (t, J=6.05 Hz, 1H) 7.00 (d, J=8.79 Hz, 1H) 7.28-7.40 (m, 3H) 8.27 (s, 1H) 8.70 (s, 1H) 12.01 (s, 1H); LCMS: 368 (M+H).

Results of the biochemical assay described above: GRK-2 ++.

### Example 8

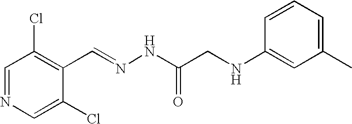

m-Tolylamino-acetic acid [1-(3,5-dichloro-pyridin-4-yl)-meth-(E)-ylidene]-hydrazide: In a similar fashion by making the appropriate substitution for the aniline, the following compound was prepared according to Example 1: ¹H NMR (400 MHz, DMSO-d₆) δ ppm 2.14 (s, 3H) 4.17 (d, J=6.05 Hz, 2H) 5.61-5.67 (m, 1H) 6.31-6.42 (m, 3H) 6.93 (t, J=7.91 Hz, 1H) 8.26 (s, 1H) 8.68-8.71 (m, 1H) 11.93 (s, 1H); LCMS: 337 (M+H).

Results of the biochemical assay described above: GRK-2 ++.

### Example 9

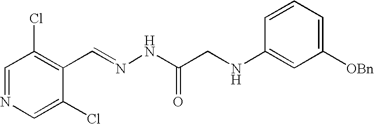

(3-Benzyloxy-phenylamino)-acetic acid [1-(3,5-dichloro-pyridin-4-yl)-meth-(E)-ylidene]-hydrazide: In a similar fashion by making the appropriate substitution for the aniline the following compound was prepared according to Example 1: ¹H NMR (400 MHz, DMSO-d₆) δ ppm 4.13-4.21 (m, 2H) 4.97 (s, 2H) 5.76-5.83 (m, 1H) 6.13-6.25 (m, 3H) 6.91-6.99 (m, 1H) 7.24-7.42 (m, 5 H) 8.25 (s, 1H) 8.67 (s, 2H) 11.94 (s, 1H); LCMS: # (M+H).

Results of the biochemical assay described above:

| | |
|---|---|
| GRK-2 | +++ |
| GRK-3 | +++ |
| GRK-5 | ++ |
| GRK-6 | +. |

### Example 10

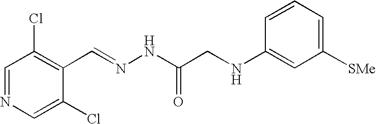

(3-Methylsulfanyl-phenylamino)-acetic acid [1-(3,5-dichloro-pyridin-4-yl)-meth-(E)-ylidene]-hydrazide: In a similar fashion by making the appropriate substitution for the aniline, the following compound was prepared according to Example 1: ¹H NMR(400 MHz, DMSO-d₆) δ ppm 2.36 (s, 3H) 4.19 (d, J=5.86 Hz, 2H) 5.84-5.91 (m, 1H) 6.31-6.38 (m, J=1.56 Hz, 1H) 6.40-6.48 (m, 2H) 6.99 (t, J=7.81 Hz, 1H) 8.26 (s, 1H) 8.68-8.71 (m, 1H) 11.96 (s, 1H); LCMS: 369 (M+H).

Results of the biochemical assay described above:

| | |
|---|---|
| GRK-2 | +++ |
| GRK-3 | +++ |
| GRK-5 | ++ |
| GRK-6 | −−. |

### Example 11

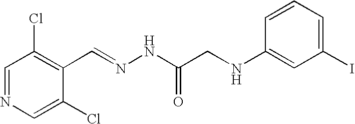

(3-Iodo-phenylamino)-acetic acid [1-(3,5-dichloro-pyridin-4-yl)-meth-(E)-ylidene]-hydrazide: In a similar fashion by making the appropriate substitution for the aniline the following compound was prepared according to Example 1: ¹H NMR (400 MHz, DMSO-d₆) δ ppm 4.18 (d, J=6.05 Hz, 2H) 6.05-6.11 (m, 1H) 6.53-6.59 (m, 1H) 6.79-6.96 (m, 3H) 8.25 (s, 1H) 8.69 (s, 1H) 11.96 (s, 1H); LCMS: 449 (M+H).

Results of the biochemical assay described above: GRK-2 ++.

### Example 12

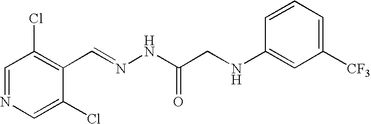

(3-Trifluoromethyl-phenylamino)-acetic acid [1-(3,5-dichloro-pyridin-4-yl)-meth-(E)-ylidene]-hydrazide: In a similar fashion by making the appropriate substitution for the aniline, the following compound was prepared according to Example 1: ¹H NMR (400 MHz, DMSO-d₆) δ ppm 4.27 (d, J=6.05 Hz, 2H) 6.33-6.40 (m, 1H) 6.78-6.88 (m, J=1.56 Hz, 4H) 7.21-7.28 (m, 1H) 8.26 (s, 1H) 8.69 (s, 1H) 11.99 (s, 1H); LCMS: 391 (M+H).

Results of the biochemical assay described above: GRK-2 +.

### Example 13

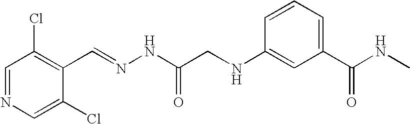

3-{[1-(3,5-Dichloro-pyridin-4-yl)-meth-(E)-ylidene-hydrazinocarbonylmethyl]-amino}-N-methyl-benzamide: In a similar fashion by making the appropriate substitution for the aniline, the following compound was prepared according to Example 1: ¹H NMR (400 MHz, DMSO-d₆) δ ppm 2.71 (d, J=4.49 Hz, 3H) 4.24 (d, J=6.05 Hz, 2H) 5.94 (t, J=6.05 Hz, 1H) 6.67-6.73 (m, 1H) 6.95-7.00 (m, 2H) 7.10 (t, J=7.81 Hz, 1H) 8.15-8.22 (m, J=4.30 Hz, 1H) 8.26 (s, 1H) 8.69 (s, 1H) 11.95 (s, 1H); LCMS: 380 (M+H).

Results of the biochemical assay described above:

| | |
|---|---|
| GRK-2 | +++ |
| GRK-3 | +++ |
| GRK-5 | ++ |
| GRK-6 | +. |

### Example 14

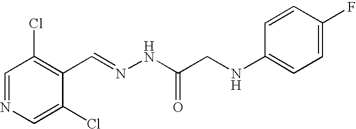

(4-Fluoro-phenylamino)-acetic acid [1-(3,5-dichloro-pyridin-4-yl)-meth-(E)-ylidene]-hydrazide: In a similar fashion by making the appropriate substitution for the aniline, the following compound was prepared according to Example 1: ¹H NMR (400 MHz, DMSO-d₆) δ ppm 4.16 (d, J=6.05 Hz, 2H) 5.69-5.75 (m, 1H) 6.51-6.57 (m, 3H) 6.89 (t, J=8.88 Hz, 2H) 8.25 (s, 1H) 8.69 (s, 1H) 11.94 (s, 1H); LCMS: 341 (M+H).

Results of the biochemical assay described above: GRK-2 ++.

### Example 15

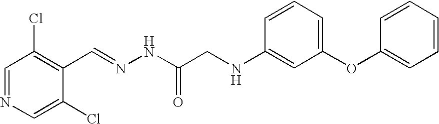

(3-Phenoxy-phenylamino)-acetic acid [1-(3,5-dichloro-pyridin-4-yl)-meth-(E)-ylidene]-hydrazide: In a similar fashion by making the appropriate substitution for the aniline, the following compound was prepared according to Example 1: ¹H NMR (400 MHz, DMSO-d₆) δ ppm 4.16 (d, J=6.05 Hz, 2H) 6.02 (s, 1H) 6.12-6.21 (m, 3H) 6.33 (d, J=8.20 Hz, 1H) 6.92-6.97 (m, J=7.81 Hz, 2 7.00-7.08 (m, 3H) 7.28-7.34 (m, 2H) 8.23 (s, 1H) 8.67 (s, 2H) 11.93 (s, 1H); LCMS: 415 (M+H).

Results of the biochemical assay described above:

| | |
|---|---|
| GRK-2 | +++ |
| GRK-3 | +++ |
| GRK-5 | ++ |
| GRK-6 | +. |

### Example 16

(3-Isopropyl-phenylamino)-acetic acid [1-(3,5-dichloro-pyridin-4-yl)-meth-(E)-ylidene]-hydrazide: In a similar fashion by making the appropriate 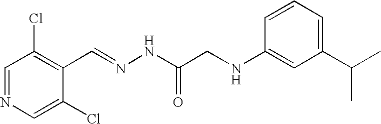
substitution for the aniline, the following compound was prepared according to Example 1: ¹H NMR (400 MHz, DMSO-d₆) δ ppm 1.12 (d, J=6.83 Hz, 6H) 2.65-2.74 (m, 1H) 4.18 (d, J=5.86 Hz, 2H) 5.63-5.68 (m, 1H) 6.32-6.49 (m, 4H) 6.96 (t, J=7.71 Hz, 1H) 8.27 (s, 1 H) 8.69 (s, 1H) 11.94 (s, 1H); LCMS: 365 (M+H).

Results of the biochemical assay described above: GRK-2+.

### Example 17

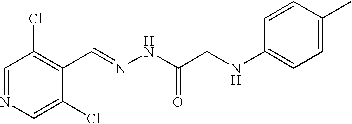

p-Tolylamino-acetic acid [1-(3,5-dichloro-pyridin-4-yl)-meth-(E)-ylidene]-hydrazide: In a similar fashion by making the appropriate substitution for the aniline, the following compound was prepared according to Example 1: ¹H NMR (400 MHz, DMSO-d₆) δ ppm 2.12 (s, 3H) 4.15 (d, J=6.05 Hz, 2H) 5.51 (t, J=5.95 Hz, 1H) 6.46 (d, J=8.40 Hz, 2H) 6.86 (d, J=8.20 Hz, 2H) 8.25 (s, 1H) 8.69 (s, 1H) 11.93 (s, 1H); LCMS: 337 (M+H).

Results of the biochemical assay described above: GRK-2 ++.

### Example 18

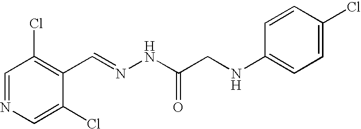

(4-Chloro-phenylamino)-acetic acid [1-(3,5-dichloro-pyridin-4-yl)-meth-(E)-ylidene]-hydrazide: In a similar fashion by making the appropriate substitution for the aniline, the following compound was prepared according to Example 1: ¹H NMR (400 MHz, DMSO-d₆) δ ppm 4.18 (d, J=6.05 Hz, 2H) 6.00 (t, J=6.05 Hz, 1H) 6.55 (d, J=8.79 Hz, 2H) 7.07 (d, J=8.79 Hz, 2H) 8.25 (s, 1H) 8.68 (s, 1H) 11.96 (s, 1H); LCMS: 357 (M+H).

Results of the biochemical assay described above: GRK-2 ++.

### Example 19

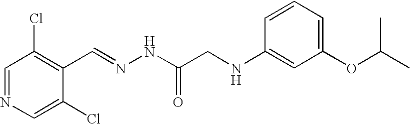

(3-Isopropoxy-phenylamino)-acetic acid [1-(3,5-dichloro-pyridin-4-yl)-meth-(E)-ylidene]-hydrazide: In a similar fashion by making the appropriate substitution for the aniline, the following compound was prepared according to Example 1: ¹H NMR (400 MHz, DMSO-d₆) δ ppm 1.18 (d, J=6.05 Hz, 6H) 4.16 (d, J=6.05 Hz, 2H) 4.39-4.49 (m, 1H) 5.72-5.77 (m, 1H) 6.06-6.17 (m, 4H) 6.92 (t, J=7.91 Hz, 1H) 8.26 (s, 1H) 8.69 (s, 1H) 11.95 (s, 1H); LCMS: 381 (M+H).

Results of the biochemical assay described above: GRK-2 ++.

### Example 20

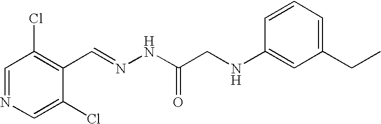

(3-Ethyl-phenylamino)-acetic acid [1-(3,5-dichloro-pyridin-4-yl)-meth-(E)-ylidene]-hydrazide: In a similar fashion by making the appropriate substitution for the aniline, the following compound was prepared according to Example 1: ¹H NMR (400 MHz, DMSO-d₆) δ ppm 1.10 (t, J=7.61 Hz, 3H) 2.39-2.47 (m, 2H) 4.18 (d, J=6.05 Hz, 2H) 5.62-5.68 (m, 1H) 6.32-6.46 (m, 4H) 6.95 (t, J=7.71 Hz, 1H) 8.26 (s, 1H) 8.69 (s, 1H) 11.94 (s, 1H); LCMS: 350 (M+H).

Results of the biochemical assay described above: GRK-2 +.

### Example 21

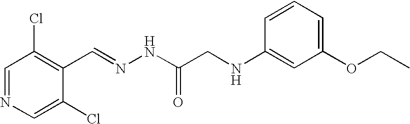

(3-Ethoxy-phenylamino)-acetic acid [1-(3,5-dichloro-pyridin-4-yl)-meth-(E)-ylidene]-hydrazide: In a similar fashion by making the appropriate substitution for the aniline, the following compound was prepared according to Example 1: ¹H NMR (400 MHz, DMSO-d₆) δ ppm 1.25 (t, J=6.93 Hz, 3H) 3.89 (q, J=6.90 Hz, 2H) 4.17 (d, J=6.05 Hz, 2H) 5.76 (t, J=5.95 Hz, 1H) 6.08-6.18 (m, 3H) 6.93 (t, J=7.91 Hz, 1H) 8.26 (s, 1H) 8.69 (s, 1H) 11.95 (s, 1H); LCMS: 367 (M+H).

Results of the biochemical assay described above:

| | |
|---|---|
| GRK-2 | +++ |
| GRK-3 | + |
| GRK-5 | + |
| GRK-6 | −−. |

### Example 22

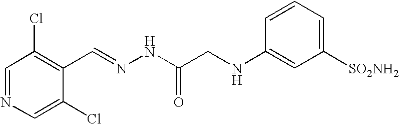

3-{[1-(3,5-Dichloro-pyridin-4-yl)-meth-(E)-ylidene-hydrazinocarbonylmethyl]-amino}-benzenesulfonamide: In a similar fashion by making the appropriate substitution for the aniline, the following compound was prepared according to Example 1: ¹H NMR (400 MHz, DMSO-d₆) δ ppm 4.25 (d, J=5.86 Hz, 2H) 4.32 (t, J=5.08 Hz, 1H) 6.28-6.32 (m, 1H) 6.74 (dd, J=8.69, 1.85 Hz, 1H) 6.96-7.01 (m, 2H) 7.15 (s, 2H) 7.18-7.24 (m, 1H) 8.27 (s, 1H) 8.69 (s, 1H) 11.98 (s, 1H); LCMS: 402 (M+H).

Results of the biochemical assay described above:

| | |
|---|---|
| GRK-2 | +++ |
| GRK-3 | ++ |
| GRK-5 | ++ |
| GRK-6 | −−. |

### Example 23

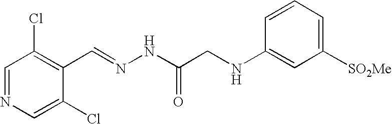

(3-Methanesulfonyl-phenylamino)-acetic acid [1-(3,5-dichloro-pyridin-4-yl)-meth-(E)-ylidene]-hydrazide: In a similar fashion by making the appropriate substitution for the aniline, the following compound was prepared according to Example 1: ¹H NMR (400 MHz, DMSO-d₆) δ ppm 4.25 (d, J=5.86 Hz, 2H) 6.27-6.33 (m, 1H) 6.74 (dd, J=8.69, 1.85 Hz, 1H) 6.99 (s, 1H) 7.15 (s, 2H) 7.17-7.26 (m, 2H) 8.27 (s, 1H) 8.69 (s, 1H) 11.98 (s, 1H); LCMS: 401 (M+H).

Results of the biochemical assay described above: GRK-2 ++.

### Example 24

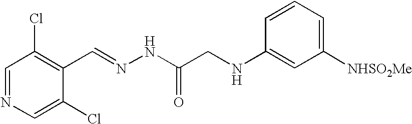

(3-Amino-ethanesulfonyl-phenylamino)-acetic acid [1-(3,5-dichloro-pyridin-4-yl)-meth-(E)-ylidene]-hydrazide: In a similar fashion by making the appropriate substitution for the aniline, the following compound was prepared according to Example 1: ¹H NMR (400 MHz, DMSO-d₆) δ ppm 2.93 (s, 3H) 4.19 (d, J=5.8 Hz, 2H) 5.81-5.98 (m, 1H) 6.27-6.56 (m, 3H) 6.93-7.10 (m, 1H) 8.29 (s, 1H), 8.71 (s, 1H); LCMS: 416 (M+H).

Results of the biochemical assay described above: GRK-2 ++.

### Example 25

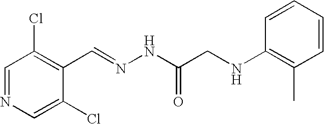

o-Tolylamino-acetic acid [1-(3,5-dichloro-pyridin-4-yl)-meth-(E)-ylidene]-hydrazide: In a similar fashion by making the appropriate substitution for the aniline, the following compound was prepared according to Example 1: ¹H NMR (400 MHz, DMSO-d₆) δ ppm 2.10 (s, 3H) 4.24 (d, J=5.86 Hz, 2H) 5.00-5.06 (m, 1H 6.34 (d, J=8.20 Hz, 1H) 6.52 (t, J=7.22 Hz, 1H) 6.94-7.01 (m, 2H) 8.28 (s, 1H) 8.70 (s, 1H) 12.02 (s, 1H); LCMS: 337 (M+H).

Results of the biochemical assay described above: GRK-2 ++.

### Example 26

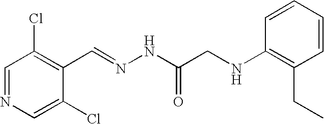

(2-Ethyl-phenylamino)-acetic acid [1-(3,5-dichloro-pyridin-4-yl)-ylidene]-hydrazide: In a similar fashion by making the appropriate substitution for the aniline, the following compound was prepared according to Example 1: ¹H NMR (400 MHz, DMSO-d₆) δ ppm 1.12-1.20 (m, 3H) 2.43-2.53 (m, 2H) 4.24 (d, J=5.66 Hz, 2H) 5.05-5.14 (m, 1H) 6.36 (d, J=8.20 Hz, 1H) 6.56 (t, J=7.22 Hz, 1H) 6.92-7.02 (m, 2H) 8.28 (s, 1H) 8.69-8.71 (m, 1H) 12.02 (s, 1H); LCMS: 351 (M+H).

Results of the biochemical assay described above: GRK-2 ++.

### Example 27

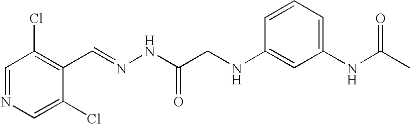

N-(3-{[1-(3,5-Dichloro-pyridin-4-yl)-meth-(E)-ylidene-hydrazinocarbonylmethyl]-amino}-phenyl)-acetamide: In a similar fashion by making the appropriate substitution for the aniline, the following compound was prepared according to Example 1: ¹H NMR (400 MHz, DMSO-d₆) δ ppm 1.96 (s, 3H) 4.15 (d, J=5.86 Hz, 2H) 5.71-5.76 (m, 1H) 6.25 (dd, J=7.71, 1.46 Hz, 1H) 6.75 (d, J=7.22 Hz, 1H) 6.86 (s, 1H) 6.93 (t, J=8.00 Hz, 1H) 8.26 (s, 1H) 8.69 (s, 1H) 11.93 (s, 1H); LCMS: 380 (M+H).

Results of the biochemical assay described above: GRK-2 ++.

### Example 28

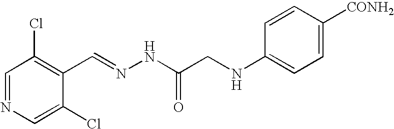

4-{[1-(3,5-Dichloro-pyridin-4-yl)-meth-(E)-ylidene-hydrazinocarbonylmethyl]-amino}-benzamide: In a similar fashion by making the appropriate substitution for the aniline, the following compound was prepared according to Example 1: ¹H NMR (400 MHz, DMSO-d₆) δ ppm 4.26 (d, J=6.05 Hz, 2H) 6.33 (t, J=5.95 Hz, 1H) 6.50-6.59 (m, 2H) 6.85 (s, 1H) 7.54 (s, 1H) 7.62 (d, J=8.79 Hz, 2H) 8.27 (s, 1H) 8.70 (s, 1H) 11.99 (s, 1H); LCMS: 366 (M+H).

Results of the biochemical assay described above: GRK-2 +.

### Example 29

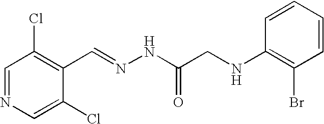

(2-Bromo-phenylamino)-acetic acid [1-(3,5-dichloro-pyridin-4-yl)-meth-(E)-ylidene]-hydrazide: In a similar fashion by making the appropriate substitution for the aniline, the following compound was prepared according to Example 1: ¹H NMR (400 MHz, DMSO-d₆) δ ppm 4.29 (d, J=5.47 Hz, 2H) 5.45 (t, J=5.37 Hz, 1H) 6.49-6.58 (m, 2H) 7.13-7.20 (m, 1H) 7.42 (dd, J=7.81, 1.37 Hz, 1H) 8.27 (s, 1H) 8.70 (s, 1H) 12.09 (s, 1H); LCMS: 401 (M+H).

Results of the biochemical assay described above: GRK-2 +.

### Example 30

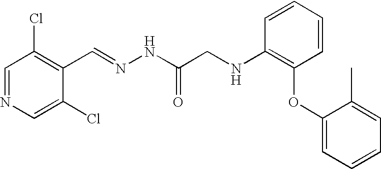

(²-o-Tolyloxy-phenylamino)-acetic acid [1-(3,5-dichloro-pyridin-4-yl)-meth-(E)-ylidene]-hydrazide: In a similar fashion by making the appropriate substitution for the aniline, the following compound was prepared according to Example 1: ¹H NMR (400 MHz, DMSO-d₆) δ ppm 2.23 (s, 3H) 4.26 (d, J=5.86 Hz, 2H) 5.33-5.39 (m, 1H) 6.52-6.63 (m, 3H) 6.71 (d, J=8.00 Hz, 1H) 6.91 -7.02 (m, 2H) 7.08-7.15 (m, 1H) 7.21-7.33 (m, 1H) 8.25 (s, 1H) 8.69 (s, 1H) 11.99 (s, 1H); LCMS: 429 (M+H).

Results of the biochemical assay described above: GRK-2 +.

### Example 31

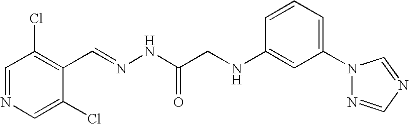

(3-[1,2,4]Triazol-1-yl-phenylamino)-acetic acid [1-(3,5-dichloro-pyridin-4-yl)-meth-(E)-ylidene]-hydrazide: In a similar fashion by making the appropriate substitution for the aniline, the following compound was prepared according to Example 1: ¹H NMR (400 MHz, DMSO-d₆) δ ppm 4.29 (d, J=5.86 Hz, 2H) 6.19-6.27 (m, 1H) 6.61 (dd, J=8.10, 1.66 Hz, 1H) 6.95-7.08 (m, 2H) 7.17-7.27 (m, J=8.00, 8.00 Hz, 1H) 8.13 (s, 1H) 8.27 (s, 1H) 8.70 (s, 1H) 9.13 (s, 1H) 11.98 (s, 1H); LCMS: 390 (M+H).

Results of the biochemical assay described above: GRK-2 +.

### Example 32

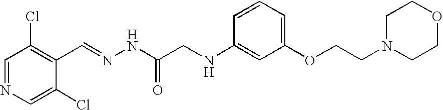

[3-(2-Morpholin-4-yl-ethoxy)-phenylamino]-acetic acid [1-(3,5-dichloro-pyridin-4-yl)-meth-(E)-ylidene]-hydrazide: In a similar fashion by making the appropriate substitution for the aniline, the following compound was prepared according to Example 1: ¹H NMR (400 MHz, DMSO-d₆) δ ppm 2.37-2.45 (m, 4H) 2.61 (t, J=5.86 Hz, 2H) 3.50-3.59 (m, 4H) 3.95 (t, J=5.76 Hz, 2H) 4.18 (d, J=6.05 Hz, 2H) 5.73-5.83 (m, 1H) 6.08-6.22 (m, 3H) 6.93 (t, J=7.91 Hz, 1H) 8.26 (s, 1H) 8.69 (s, 2H) 11.95 (s, 1H); LCMS: 452 (M+H).

Results of the biochemical assay described above: GRK-2 ++.

### Example 33

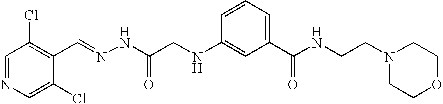

3-{[1-(3,5-Dichloro-pyridin-4-yl)-meth-(E)-ylidene-hydrazinocarbonylmethyl]-amino}-N-(2-morpholin-4-yl-ethyl)-benzamide: In a similar fashion by making the appropriate substitution for the aniline, the following compound was prepared according to Example 1: ¹H NMR (400 MHz, DMSO-d₆) 6 ppm 2.33-2.44 (m, 4H) 3.24-3.35 (m, 4H) 3.49-3.56 (m, 5 H) 4.20-4.26 (m, 2H) 6.68-6.73 (m, 1H) 6.93-7.00 (m, 2H) 7.07-7.15 (m, 1H) 8.10-8.17 (m, 1H) 8.26 (s, 1H) 8.68 (s, 2H) 11.94 (s, 1H); LCMS: 479 (M+H).

Results of the biochemical assay described above: GRK-2 ++++.

### Example 34

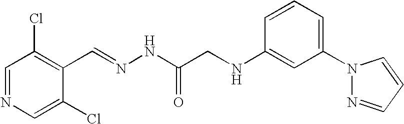

(3-Pyrazol-1-yl-phenylamino)-acetic acid [1-(3,5-Dichlro-pyridin-4-yl-meth-(E)-ylidene]-hydrazide: In a similar fashion by making the appropriate substitution for the aniline, the following compound was prepared according to Example 1: ¹H NMR (400 MHz, DMSO-d₆) δ ppm 4.27 (d, J=6.05 Hz, 2H) 6.05-6.12 (m, 1H) 6.43-6.54 (m, 2H) 6.93-6.97 (m, 1H) 7.02-7.08 (m, J=1.95, 1.95 Hz, 1H) 7.14 (t, J=8.00 Hz, 1H) 7.61-7.68 (m, J=1.56 Hz, 1H) 8.24-8.34 (m, 2H) 8.65-8.71 (m, 2H) 11.97 (s, 1H); LCMS: 389 (M+H).

Results of the biochemical assay described above: GRK-2 +.

### Example 35

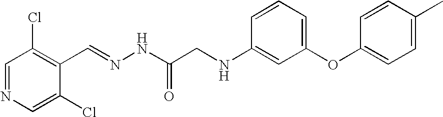

(3-p-Tolyloxy-phenylamino)-acetic acid [1-(3,5-dichloro-pyridin-4-yl)-meth-(E)-ylidene]-hydrazide: In a similar fashion by making the appropriate substitution for the aniline, the following compound was prepared according to Example 1: ¹H NMR (400 MHz, DMSO-d₆) δ ppm 2.23 (s, 3H) 4.14 (d, J=6.44 Hz, 2H) 5.98 (t, J=6.64 Hz, 1H) 6.09-6.16 (m, 2H) 6.30 (d, J=7.42 Hz, 1H) 6.85 (d, J=8.40 Hz, 2H) 7.01 (t, J=8.00 Hz, 1H) 7.11 (d, J=8.20 Hz, 2H) 8.23 (s, 1H) 8.67 (s, 1H); LCMS: 429 (M+H).

Results of the biochemical assay described above: GRK-2 ++.

### Example 36

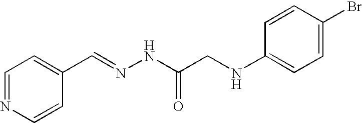

(4-Bromo-phenylamino)-acetic acid [1-pyridin-4-yl-meth-(E)-ylidene]-hydrazide: In a similar fashion by making the appropriate substitution for the aniline in Example 1, Step 1, and substituting 4-pyridinecarboxaldehyde for 3,5-dichloro-4-pyridinecarboxaldehyde in Example 1, Step 3, the following compound was prepared: ¹H NMR (400 MHz, DMSO-d₆) δ ppm 4.23 (d, J=6.05 Hz, 2H) 6.00 (t, J=5.95 Hz, 1H) 6.56-6.61 (m, 2H) 7.18 (d, J=8.98 Hz, 2H) 7.64-7.68 (m, 2H) 7.96 (s, 1H) 8.59-8.63 (m, 2H) 11.74 (s, 1H); LCMS: 333 (M+H).

Results of the biochemical assay described above: GRK-2 ++.

### Example 37

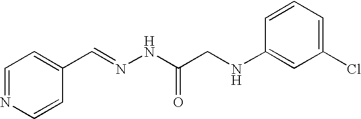

(3-Chloro-phenylamino)-acetic acid [1-pyridin-4-yl-meth-(E)-ylidene]-hydrazide: In a similar fashion by making the appropriate substitution for the aniline in Example 1, Step 1, and substituting 4-pyridinecarboxaldehyde for 3,5-dichloro-4-pyridinecarboxaldehyde in Example 1, Step 3, the following compound was prepared: ¹H NMR (400 MHz, chloroform-d) δ ppm 4.35 (d, J=5.08 Hz, 2H) 4.73 (t, J=4.98 Hz, 1H) 6.57 (d, J=8.00 Hz, 1H) 6.67 (s, 1H) 6.72 (d, J=7.81 Hz, 1H) 7.11 (t, J=8.00 Hz, 1H) 7.55 (d, J=6.05 Hz, 2H) 7.74 (s, 1H) 8.70-8.76 (m, 2H) 9.19 (s, 1H); LCMS: 289 (M+H).

Results of the biochemical assay described above:

| | |
|---|---|
| GRK-2 | +++ |
| GRK-3 | ++ |
| GRK-5 | + |
| GRK-6 | −−. |

### Example 38

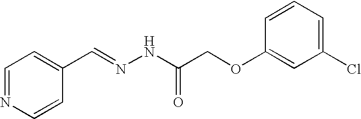

(3-Chloro-phenoxy)-acetic acid [1-pyridin-4-yl-meth-(E)-ylidene]-hydrazide: To a suspension of NaH (0.25 g, 10.5 mmol) in THF at 0° C. was added 3-chlorophenol (1 mL, 9.5 mmol). The reaction mixture was stirred for 30 min then ethyl bromoacetate (1.05 mL, 9.5 mmol) was added and the reaction stirred at 25° C. for 72 h before being quenched with water and diluted with EtOAc. The organic layer was dried with Na₂SO₄, filtered and concentrated to provide ester. This compound was treated with hydrazine as in Example 1, Step 2, and then as in Example 1, Step 3, by substituting 4-pyridinecarboxaldehyde for 3,5-dichloro-4-pyridinecarboxaldehyde: ¹H NMR (400 MHz, DMSO-d₆) δ ppm 5.22 (s, 2H) 6.89-7.09 (m, 4H) 7.26-7.36 (m, 1H) 7.62 (d, J=5.86 Hz, 1H) 7.64-7.68 (m, 2H) 7.97 (s, 1H) 8.60-8.65 (m, 2H) 11.84 (s, 1H); LCMS: 290 (M+H).

Results of the biochemical assay described above: GRK-2 +.

### Example 39

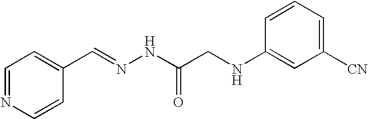

(3-Cyano-phenylamino)-acetic acid [1-pyridin-4-yl-meth-(E)-ylidene]-hydrazide: In a similar fashion by making the appropriate substitution for the aniline in Example 1, Step 1, and substituting 4-pyridinecarboxaldehyde for 3,5-dichloro-4-pyridinecarboxaldehyde in Example 1, Step 3, the following compound was prepared: ¹H NMR (400 MHz, DMSO-d₆) δ ppm 4.31 (d, J=6.05 Hz, 2H) 6.33 (t, J=5.76 Hz, 1H) 6.87-6.99 (m, 3H) 7.24 (t, J=7.81 Hz, 1H) 7.68 (dd, J=4.59, 1.46 Hz, 2H) 7.97 (s, 1H) 8.59-8.64 (m, 2H) 11.78 (s, 1H); LCMS: 280 (M+H).

Results of the biochemical assay described above: GRK-2 ++.

### Example 40

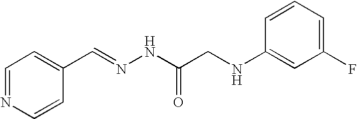

(3-Fluoro-phenylamino)-acetic acid [1-pyridin-4-yl-meth-(E)-ylidene]-hydrazide: In a similar fashion by making the appropriate substitution for the aniline in Example 1, Step 1, and substituting 4-pyridinecarboxaldehyde for 3,5-dichloro-4-pyridinecarboxaldehyde in Example 1, Step 3, the following compound was prepared: ¹H NMR (400 MHz, DMSO-d₆) δ ppm 4.20 (d, J=6.05 Hz, 2H) 6.12-6.19 (m, 1H) 6.25-6.42 (m, 4H) 7.05 (q, J=7.87 Hz, 1H) 8.26 (s, 1H) 8.69 (s, 2H) 11.97 (s, 1H); LCMS: 273 (M+H).

Results of the biochemical assay described above:

| | |
|---|---|
| GRK-2 | +++ |
| GRK-3 | ++ |
| GRK-5 | + |
| GRK-6 | −−. |

### Example 41

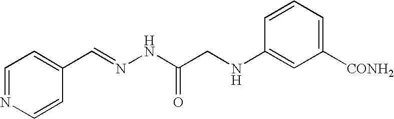

3-{[1-Pyridin-4-yl-meth-(E)-ylidene-hydrazinocarbonylmethyl]-amino}-benzamide: In a similar fashion by making the appropriate substitution for the aniline in Example 1, Step 1, and substituting 4-pyridinecarboxaldehyde for 3,5-dichloro-4-pyridinecarboxaldehyde in Example 1, Step 3, the following compound was prepared: ¹H NMR (400 MHz, DMSO-d₆) δ ppm 4.24 (d, J=6.05 Hz, 2H) 5.87-5.94 (m, 1H) 6.71 (d, J=5.47 Hz, 1H) 6.99-7.17 (m, 5 H) 7.74 (s, 1H) 8.24-8.28 (m, 1H) 8.65-8.71 (m, 3H) 11.95 (s, 1H); LCMS: 366 (M+H).

Results of the biochemical assay described above:

| | |
|---|---|
| GRK-2 | ++++ |
| GRK-3 | ++++ |
| GRK-5 | +++ |
| GRK-6 | +. |

### Example 42

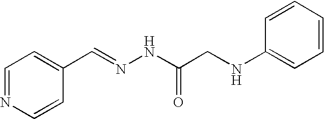

Phenylamino-acetic acid [1-pyridin-4-yl-meth-(E)-ylidene]-hydrazide: In a similar fashion by making the appropriate substitution for the aniline in Example 1, Step 1, and substituting 4-pyridinecarboxaldehyde for 3,5-dichloro-4-pyridinecarboxaldehyde in Example 1, Step 3, the following compound was prepared: ¹H NMR (400 MHz, DMSO-d₆) δ ppm 4.19 (d, J=6.05 Hz, 2H) 5.74 (t, J=5.95 Hz, 1H) 6.49-6.60 (m, 4H) 7.05 (t, J=7.81 Hz, 2H) 8.26 (s, 1H) 8.69 (s, 2H) 11.95 (s, 1H); LCMS: 255 (M+H).

Results of the biochemical assay described above: GRK-2 +++.

### Example 43

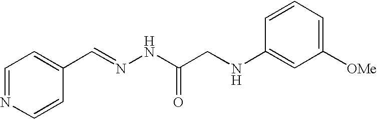

(3-Methoxy-phenylamino)-acetic acid [1-pyridin-4-yl-meth-(E)-ylidene]-hydrazide: In a similar fashion by making the appropriate substitution for the aniline in Example 1, Step 1, and substituting 4-pyridinecarboxaldehyde for 3,5-dichloro-4-pyridinecarboxaldehyde in Example 1, Step 3, the following compound was prepared: ¹H NMR (400 MHz, DMSO-d₆) δ ppm 3.63 (s, 3H) 4.17 (d, J=6.05 Hz, 2H) 5.77 (t, J=5.86 Hz, 1H) 6.09-6.19 (m, 3H) 6.94 (t, J=7.91 Hz, 1H) 8.26 (s, 1H) 8.69 (s, 2H) 11.95 (s, 3H); LCMS: 285 (M+H).

Results of the biochemical assay described above:

| | |
|---|---|
| GRK-2 | ++++ |
| GRK-3 | ++++ |
| GRK-5 | ++ |
| GRK-6 | +. |

### Example 44

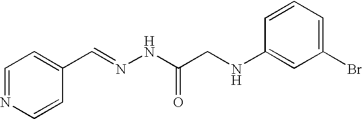

(3-Bromo-phenylamino)-acetic acid [1-pyridin-4-yl-meth-(E)-ylidene]-hydrazide: In a similar fashion by making the appropriate substitution for the aniline in Example 1, Step 1, and substituting 4-pyridinecarboxaldehyde for 3,5-dichloro-4-pyridinecarboxaldehyde in Example 1, Step 3, the following compound was prepared: ¹H NMR (400 MHz, DMSO-d₆) δ ppm 4.25 (d, J=6.05 Hz, 2H) 6.12 (t, J=6.05 Hz, 1H) 6.60 (dd, J=8.30, 1.66 Hz, 1H) 6.63-6.75 (m, 1H) 6.79 (t, J=2.05 Hz, 1H) 6.99 (t, J=7.91 Hz, 1H) 7.67 (dd, J=4.49, 1.56 Hz, 2H) 7.97 (s, 1H) 8.58-8.64 (m, 2H) 11.75 (s, 1H); LCMS: 333 (M+H).

Results of the biochemical assay described above: GRK-2 +++.

### Example 45

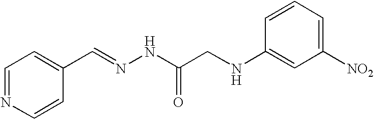

(3-Nitro-phenylamino)-acetic acid [1-pyridin-4-yl-meth-(E)-ylidene]-hydrazide: In a similar fashion by making the appropriate substitution for the aniline in Example 1, Step 1, and substituting 4-pyridinecarboxaldehyde for 3,5-dichloro-4-pyridinecarboxaldehyde in Example 1, Step 3, the following compound was prepared: ¹H NMR (400 MHz, DMSO-d₆) δ ppm 4.37 (d, J=6.05 Hz, 2H) 6.58-6.64 (m, 1H) 7.02-7.06 (m, 1H) 7.29-7.38 (m, 3H) 7.41 (t, J=2.05 Hz, 1H) 7.68 (dd, J=4.59, 1.46 Hz, 2H) 7.98 (s, 1H) 8.59-8.64 (m, 2H) 11.80 (s, 1H); LCMS: 300 (M+H).

### Example 46

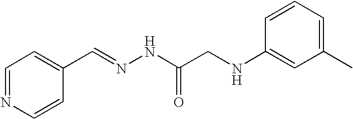

(3-Methyl-phenylamino)-acetic acid [1-pyridin-4-yl-meth-(E)-ylidene]-hydrazide: In a similar fashion by making the appropriate substitution for the aniline in Example 1, Step 1, and substituting 4-pyridinecarboxaldehyde for 3,5-dichloro-4-pyridinecarboxaldehyde in Example 1, Step 3, the following compound was prepared: ¹H NMR (400 MHz, DMSO-d₆) δ ppm 2.16 (s, 3H) 4.22 (d, J=5.86 Hz, 2H) 5.60 (t, J=5.86 Hz, 1H) 6.33-6.44 (m, 3H) 6.93 (t, J=7.52 Hz, 1H) 7.66 (dd, J=4.59, 1.46 Hz, 2H) 7.97 (s, 1H) 8.58-8.64 (m, 2H) 11.67-11.72 (m, J=0.78 Hz, 1H); LCMS: 269 (M+H).

Results of the biochemical assay described above: GRK-2 ++.

### Example 47

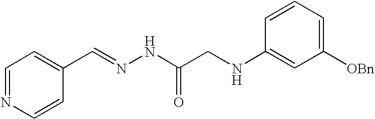

(3-Benzyloxy-phenylamino)-acetic acid [1-pyridin-4-yl-meth-(E)-ylidene]-hydrazide: In a similar fashion by making the appropriate substitution for the aniline in Example 1, Step 1, and substituting 4-pyridinecarboxaldehyde for 3,5-dichloro-4-pyridinecarboxaldehyde in Example 1, Step 3, the following compound was prepared: ¹H NMR (400 MHz, DMSO-d₆) δ ppm 4.21 (d, J=5.86 Hz, 2H) 4.98 (s, 2H) 5.75 (t, J=5.76 Hz, 1H) 6.18-6.27 (m, 3H) 6.92-6.97 (m, 1H) 7.24-7.41 (m, 5 H) 7.66 (dd, J=4.59, 1.46 Hz, 2H) 7.97 (s, 1H) 8.58-8.63 (m, 2H) 11.74 (s, 1H); LCMS: 360 (M+).

Results of the biochemical assay described above:

| | |
|---|---|
| GRK-2 | +++ |
| GRK-3 | +++ |
| GRK-5 | ++ |
| GRK-6 | +. |

### Example 48

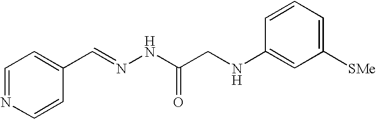

(3-Methylsulfanyl-phenylamino)-acetic acid [1-pyridin-4-yl-meth-(E)-ylidene]-hydrazide: In a similar fashion by making the appropriate substitution for the aniline in Example 1, Step 1, and substituting 4-pyridinecarboxaldehyde for 3,5-dichloro-4-pyridinecarboxaldehyde in Example 1, Step 3, the following compound was prepared: ¹H NMR (400 MHz, DMSO-d₆) δ ppm 2.36 (s, 3H) 4.24 (d, J=5.86 Hz, 2H) 5.85 (t, J=5.86 Hz, 1H) 6.32-6.48 (m, 3H) 6.51 (t, J=1.85 Hz, 1H) 6.96-7.01 (m, 1H) 7.66 (dd, J=4.59, 1.46 Hz, 2H) 7.97 (s, 1H) 8.58-8.63 (m, 2H) 11.73 (s, 1H); LCMS: 301 (M+H).

Results of the biochemical assay described above:

| | |
|---|---|
| GRK-2 | +++ |
| GRK-3 | +++ |
| GRK-5 | + |
| GRK-6 | −−. |

### Example 49

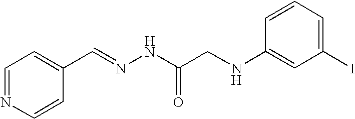

(3-Iodo-phenylamino)-acetic acid [1-pyridin-4-yl-meth-(E)-ylidene]-hydrazide: In a similar fashion by making the appropriate substitution for the aniline in Example 1, Step 1, and substituting 4-pyridinecarboxaldehyde for 3,5-dichloro-4-pyridinecarboxaldehyde in Example 1, Step 3, the following compound was prepared: ¹H NMR (400 MHz, DMSO-d₆) δ ppm 4.23 (d, J=6.05 Hz, 2H) 6.04 (s, 1H) 6.58-6.64 (m, 1H) 6.81-6.85 (m, 2H) 6.96-7.00 (m, J=1.56 Hz, 1H) 7.66 (dd, J=4.49, 1.37 Hz, 2H) 7.97 (s, 1H) 8.58-8.64 (m, J=4.49, 1.56 Hz, 2H) 11.75 (s, 1H); LCMS: 381 (M+H).

Results of the biochemical assay described above: GRK-2 +++.

### Example 50

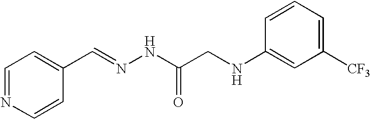

(3-Trifluoromethyl-phenylamino)-acetic acid [1-pyridin-4-yl-meth-(E)-ylidene]-hydrazide: In a similar fashion by making the appropriate substitution for the aniline in Example 1, Step 1, and substituting 4-pyridinecarboxaldehyde for 3,5-dichloro-4-pyridinecarboxaldehyde in Example 1, Step 3, the following compound was prepared: ¹H NMR (400 MHz, DMSO-d₆) δ ppm 4.32 (d, J=6.05 Hz, 2H) 6.33 (t, J=5.95 Hz, 1H) 6.79-6.87 (m, J=14.35, 7.71 Hz, 2H) 6.91 (s, 1H) 7.26 (t, J=7.81 Hz, 1H) 7.67 (dd, J=4.59, 1.46 Hz, 2H) 7.97 (s, 1H) 8.59-8.64 (m, 2H) 11.77 (s, 1H); LCMS: 323 (M+H).

Results of the biochemical assay described above: GRK-2 ++.

### Example 51

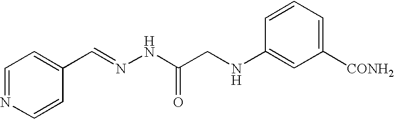

3-{[1-Pyridin-4-yl-meth-(E)-ylidene-hydrazinocarbonylmethyl]-amino}-benzamide: In a similar fashion by making the appropriate substitution for the aniline in Example 1, Step 1, and substituting 4-pyridinecarboxaldehyde for 3,5-dichloro-4-pyridinecarboxaldehyde in Example 1, Step 3, the following compound was prepared: ¹H NMR (400 MHz, DMSO-d₆) δ ppm 4.29 (d, J=6.05 Hz, 2H) 5.88 (t, J=5.86 Hz, 1H) 6.76 (d, J=7.81 Hz, 1H) 7.01-7.08 (m, 2H) 7.11 (t, J=7.71 Hz, 1H) 7.14-7.18 (m, 1H) 7.66 (dd, J=4.59, 1.46 Hz, 2H) 7.74-7.78 (m, 1H) 7.98 (s, 1H) 8.58-8.64 (m, 2H) 11.74 (s, 1H); LCMS: 298 (M+H).

Results of the biochemical assay described above:

| | |
|---|---|
| GRK-2 | +++ |
| GRK-3 | +++ |
| GRK-5 | −− |
| GRK-6 | −−. |

### Example 52

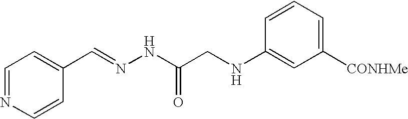

N-Methyl-3-{[1-pyridin-4-yl-meth-(E)-ylidene-hydrazinocarbonyl methyl]-amino}-benzamide: In a similar fashion by making the appropriate substitution for the aniline in Example 1, Step 1, and substituting 4-pyridinecarboxaldehyde for 3,5-dichloro-4-pyridinecarboxaldehyde in Example 1, Step 3, the following compound was prepared: ¹H NMR (400 MHz, DMSO-d₆) δ ppm 2.71 (d, J=4.49 Hz, 3H) 4.29 (d, J=6.05 Hz, 2H) 5.92 (s, 1H) 6.72-6.77 (m, J=1.76 Hz, 1H) 6.95-7.00 (m, 1H) 7.02 (d, J=2.15 Hz, 2H) 7.11 (t, J=7.81 Hz, 1H) 7.66 (dd, J=4.49, 1.37 Hz, 2H) 7.98 (s, 1H) 8.17-8.21 (m, 1H) 8.58-8.64 (m, 2H) 11.74 (s, 1H); LCMS: 312 (M+H).

Results of the biochemical assay described above:

| | |
|---|---|
| GRK-2 | +++ |
| GRK-3 | +++ |
| GRK-5 | + |
| GRK-6 | −−. |

### Example 53

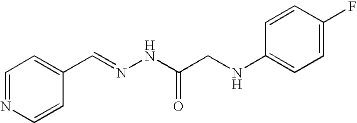

(4-fluoro-phenylamino)-acetic acid [1-pyridin-4-yl-meth-(E)-ylidene]-hydrazide: In a similar fashion by making the appropriate substitution for the aniline in Example 1, Step 1, and substituting 4-pyridinecarboxaldehyde for 3,5-dichloro-4-pyridinecarboxaldehyde in Example 1, Step 3, the following compound was prepared: ¹H NMR (400 MHz, DMSO-d₆) δ ppm 4.21 (d, J=5.86 Hz, 2H) 5.68 (t, J=5.95 Hz, 1H) 6.58-6.63 (m, 2H) 6.86-6.93 (m, 2H) 7.66 (d, J=6.05 Hz, 2H) 7.96 (s, 1H) 8.58-8.64 (m, 2H) 11.72 (s, 1H); LCMS: 273 (M+H).

Results of the biochemical assay described above: GRK-2 ++.

### Example 54

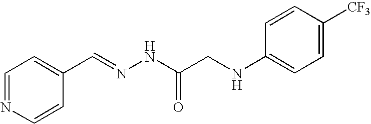

(4-Trifluoromethyl-phenylamino)-acetic acid [1-pyridin-4-yl-meth-(E)-ylidene]-hydrazide: In a similar fashion by making the appropriate substitution for the aniline in Example 1, Step 1, and substituting 4-pyridinecarboxaldehyde for 3,5-dichloro-4-pyridinecarboxaldehyde in Example 1, Step 3, the following compound was prepared: ¹H NMR (400 MHz, DMSO-d₆) δ ppm 4.32 (d, J=5.86 Hz, 2H) 6.50-6.55 (m, 1H) 6.72 (d, J=8.59 Hz, 2H) 7.33-7.38 (m, J=8.79 Hz, 2H) 7.65-7.69 (m, 2H) 7.97 (s, 1H) 8.58-8.64 (m, 2H) 11.79 (s, 1H); LCMS: 323 (M+H).

Results of the biochemical assay described above: GRK-2 +.

### Example 55

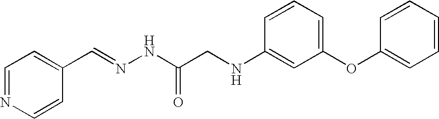

(3-Phenoxy-phenylamino)-acetic acid [1-pyridin-4-yl-meth-(E)-ylidene]-hydrazide: In a similar fashion by making the appropriate substitution for the aniline in Example 1, Step 1, and substituting 4-pyridinecarboxaldehyde for 3,5-dichloro-4-pyridinecarboxaldehyde in Example 1, Step 3, the following compound was prepared: ¹H-NMR (400 MHz, DMSO-d₆) δ ppm 4.21 (d, J=6.05 Hz, 2H) 5.95-6.01 (m, 1H) 6.11-6.19 (m, 1H) 6.27 (t, J=2.25 Hz, 1H) 6.37-6.42 (m, J=1.56 Hz, 1H) 6.92-6.97 (m, 2H) 7.01-7.09 (m, 2H) 7.28-7.35 (m, 2H) 7.62 (dd, J=4.59, 1.46 Hz, 2H) 7.94 (s, 1H) 8.58-8.63 (m, 2H) 11.74 (s, 1H); LCMS: 347 (M+H).

Results of the biochemical assay described above:

| | |
|---|---|
| GRK-2 | +++ |
| GRK-3 | +++ |
| GRK-5 | + |
| GRK-6 | +. |

### Example 56

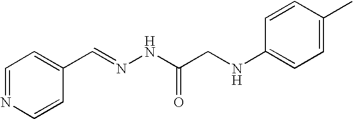

(4-Methyl-phenylamino)-acetic acid [1-pyridin-4-yl-meth-(E)-ylidene]-hydrazide: In a similar fashion by making the appropriate substitution for the aniline in Example 1, Step 1, and substituting 4-pyridinecarboxaldehyde for 3,5-dichloro-4-pyridinecarboxaldehyde in Example 1, Step 3, the following compound was prepared: ¹H NMR (400 MHz, DMSO-d₆) δ ppm 2.12 (s, 3H) 4.20 (d, J=5.47 Hz, 2H) 5.43-5.50 (m, 1H) 6.52 (d, J=8.40 Hz, 2H) 6.85-6.91 (m, 2H) 7.66 (dd, J=4.49, 1.56 Hz, 2H) 7.96 (s, 1H) 8.58-8.63 (m, 2H) 11.73 (s, 1H); LCMS: 269 (M+H).

Results of the biochemical assay described above: GRK-2 +.

### Example 57

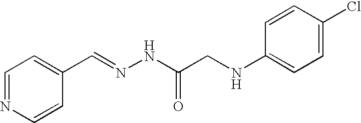

(4-Chloro-phenylamino)-acetic acid [1-pyridin-4-yl-meth-(E)-ylidene]-hydrazide: In a similar fashion by making the appropriate substitution for the aniline in Example 1, Step 1, and substituting 4-pyridinecarboxaldehyde for 3,5-dichloro-4-pyridinecarboxaldehyde in Example 1, Step 3, the following compound was prepared: ¹H NMR (400 MHz, DMSO-d₆) δ ppm 4.23 (d, J=6.05 Hz, 2H) 5.97 (t, J=5.95 Hz, 1H) 6.59-6.65 (m, 2H) 7.04-7.12 (m, 2H) 7.63-7.69 (m, 2H) 7.96 (s, ²H) 8.58-8.64 (m, 2H) 11.76 (1H); LCMS: 289 (M+H).

Results of the biochemical assay described above: GRK-2 ++.

### Example 58

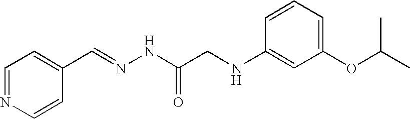

(3-Isopropoxy-phenylamino)-acetic acid [1-pyridin-4-yl-meth-(E)-ylidene]-hydrazide: In a similar fashion by making the appropriate substitution for the aniline in Example 1, Step 1, and substituting 4-pyridinecarboxaldehyde for 3,5-dichloro-4-pyridinecarboxaldehyde in Example 1, Step 3, the following compound was prepared: ¹H NMR (400 MHz, DMSO-d₆) δ ppm 1.19 (t, J=5.76 Hz, 6H) 4.21 (d, J=5.86 Hz, 2H) 4.39-4.51 (m, 1H) 5.67-5.76 (m, 1H) 6.06-6.21 (m, 3H) 6.88-6.97 (m, 1H) 7.66 (dd, J=4.49, 1.56 Hz, 2H) 7.97 (s, 1H) 8.57-8.64 (m, 2H) 11.74 (s, 1H); LCMS: 313 (M+H).

Results of the biochemical assay described above: GRK-2 ++.

### Example 59

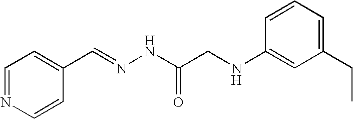

(3-Ethyl-phenylamino)-acetic acid [1-pyridin-4-yl-meth-(E)-ylidene]-hydrazide: In a similar fashion by making the appropriate substitution for the aniline in Example 1, Step 1, and substituting 4-pyridinecarboxaldehyde for 3,5-dichloro-4-pyridinecarboxaldehyde in Example 1, Step 3, the following compound was prepared: ¹H NMR (400 MHz, DMSO-d₆) δ ppm 1.10 (t, J=7.61 Hz, 3H) 2.41-2.48 (2H) 4.22 (d, J=5.27 Hz, 2H) 5.58-5.65 (m, 1H) 6.33-6.44 (m, 2H) 6.47 (s, 1H) 6.93-7.01 (m, 1H) 7.64-7.68 (m, 2H) 7.97 (s, 1H) 8.58-8.64 (m, 2H) 11.73 (s, 1H); LCMS: 283 (M+H).

Results of the biochemical assay described above: GRK-2 ++.

### Example 60

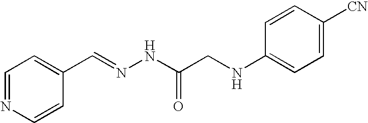

(4-Cyano-phenylamino)-acetic acid [1-pyridin-4-yl-meth-(E)-ylidene-hydrazide: In a similar fashion by making the appropriate substitution for the aniline in Example 1, Step 1, and substituting 4-pyridinecarboxaldehyde for 3,5-dichloro-4-pyridinecarboxaldehyde in Example 1, Step 3, the following compound was prepared: ¹H NMR (400 MHz, DMSO-d₆) δ ppm 4.34 (d, J=5.86 Hz, 2H) 6.71 (d, J=8.79 Hz, 2H) 6.80-6.86 (m, 1H) 7.44 (d, J=8.98 Hz, 2H) 7.67 (dd, J=4.49, 1.37 Hz, 2H) 7.98 (s, 1H) 8.57-8.64 (m, 2H) 11.95 (s, 1H); LCMS: 280 (M+H).

Results of the biochemical assay described above: GRK-2 ++.

### Example 61

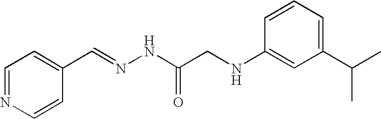

(3-Isopropyl-phenylamino)-acetic acid [1-pyridin-4-yl-meth-(E)-ylidene]-hydrazide: In a similar fashion by making the appropriate substitution for the aniline in Example 1, Step 1, and substituting 4-pyridinecarboxaldehyde for 3,5-dichloro-4-pyridinecarboxaldehyde in Example 1, Step 3, the following compound was prepared: ¹H NMR (400 MHz, DMSO-d₆) δ ppm 1.11-1.16 (m, 6H) 2.66-2.76 (m, J=7.22 Hz, 1H) 4.23 (d, J=5.86 Hz, 2H) 5.60-5.66 (m, 1H) 6.32-6.46 (m, 2H) 6.49 (d, J=14.06 Hz, 2H) 6.94-7.01 (m, 1H) 7.66 (dd, J=4.59, 1.46 Hz, 2H) 7.98 (s, 1H) 8.58-8.64 (m, 2H) 11.74 (s, 1H); LCMS: 297 (M+H).

Results of the biochemical assay described above: GRK-2 +.

### Example 62

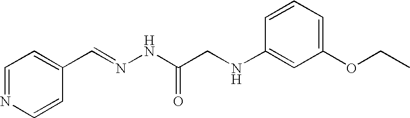

(3-Ethoxy-phenylamino)-acetic acid [1-pyridin-4-yl-meth-(E)-ylidene]-hydrazide: In a similar fashion by making the appropriate substitution for the aniline in Example 1, Step 1, and substituting 4-pyridinecarboxaldehyde for 3,5-dichloro-4-pyridinecarboxaldehyde in Example 1, Step 3, the following compound was prepared: ¹H NMR (400 MHz, DMSO-d₆) δ ppm 1.25 (q, J=7.03 Hz, 3H) 3.85-3.94 (m, 2H) 4.21 (d, J=6.05 Hz, 2H) 5.73 (t, J=5.86 Hz, 1H) 6.08-6.17 (m, 2H) 6.91-6.95 (m, 1H) 7.66 (dd, J=4.49, 1.56 Hz, 2H) 7.97 (s, 1H) 8.58-8.64 (m, 2H) 11.74 (s, 1H); LCMS: 299 (M+H).

### Example 63

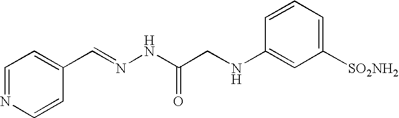

3-{[1-Pyridin-4-yl-meth-(E)-ylidene-hydrazinocarbonylmethyl]-amino}-benzenesulfonamide: In a similar fashion by making the appropriate substitution for the aniline in Example 1, Step 1, and substituting 4-pyridinecarboxaldehyde for 3,5-dichloro-4-pyridinecarboxaldehyde in Example 1, Step 3, the following compound was prepared: ¹H NMR (400 MHz, DMSO-d₆) δ ppm 4.30 (d, J=5.66 Hz, 2H) 6.25-6.32 (m, 1H) 6.78 (dd, J=8.10, 1.66 Hz, 1H) 6.95-7.06 (m, 2H) 7.13-7.28 (m, 3H) 7.64-7.69 (m, 2H) 7.98 (s, 1H) 8.58-8.65 (m, 2H) 11.77 (s, 1H); LCMS: 334 (M+H).

Results of the biochemical assay described above:

| | |
|---|---|
| GRK-2 | +++ |
| GRK-3 | ++ |
| GRK-5 | + |
| GRK-6 | −−. |

### Example 64

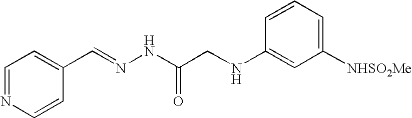

(3-Methanesulfonyl-phenylamino)-acetic acid [1-pyridin-4-yl-meth-(E)-ylidene]-hydrazide: In a similar fashion by making the appropriate substitution for the aniline in Example 1, Step 1, and substituting 4-pyridinecarboxaldehyde for 3,5-dichloro-4-pyridinecarboxaldehyde in Example 1, Step 3, the following compound was prepared: ¹H NMR (400 MHz, DMSO-d₆) δ ppm 2.92 (s, 3H) 4.21 (d, J=5.86 Hz, 2H) 5.90 (t, J=5.95 Hz, 1H) 6.35 (dd, J=7.91, 1.66 Hz, 1H) 6.39-6.45 (m, J=7.52, 1.66 Hz, 1H) 6.45-6.50 (m, J=1.95 Hz, 1H) 6.99 (t, J=7.91 Hz, 1H) 7.63-7.68 (m, 2H) 7.97 (s, 1H) 8.58-8.65 (m, 2H) 9.41 (s, 1H) 11.75 (s, 1H); LCMS: 348 (M+H).

Results of the biochemical assay described above: GRK-2 +.

### Example 65

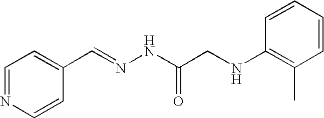

o-Tolylamino-acetic acid [1-pyridin-4-yl-meth-(E)-ylidene]-hydrazide: In a similar fashion by making the appropriate substitution for the aniline in Example 1, Step 1, and substituting 4-pyridinecarboxaldehyde for 3,5-dichloro-4-pyridinecarboxaldehyde in Example 1, Step 3, the following compound was prepared: ¹H NMR (400 MHz, DMSO-d₆) δ ppm 2.11 (s, 3H) 4.29 (d, J=5.47 Hz, 2H) 4.96-5.03 (m, 1H) 6.44-6.57 (m, 2H) 6.95-7.01 (m, J=7.22 Hz, 2H) 7.64-7.70 (m, 2H) 7.99 (s, 1H) 8.62 (d, J=6.05 Hz, 2H) 11.82 (s, 1H); LCMS: 269 (M+H).

Results of the biochemical assay described above: GRK-2 +.

### Example 66

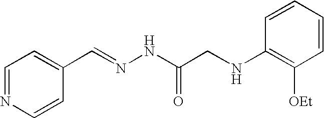

(2-Ethoxy-phenylamino)-acetic acid [1-pyridin-4-yl-meth-(E)-ylidene]-hydrazide: In a similar fashion by making the appropriate substitution for the aniline in Example 1, Step 1, and substituting 4-pyridinecarboxaldehyde for 3,5-dichloro-4-pyridinecarboxaldehyde in Example 1, Step 3, the following compound was prepared: ¹H NMR (400 MHz, DMSO-d₆) δ ppm 1.30-1.37 (m, 3H) 4.02 (q, J=6.90 Hz, 2H) 4.26 (d, J=5.66 Hz, 2H) 5.16 (t, J=5.47 Hz, 1H) 6.51-6.60 ( 2H) 6.70-6.83 (m, 2H) 7.65-7.71 (m, 2H) 7.98 (s, 1H) 8.59-8.65 (m, 2H) 11.83 1H); LCMS: 299 (M+H).

Results of the biochemical assay described above: GRK-2 +.

### Example 67

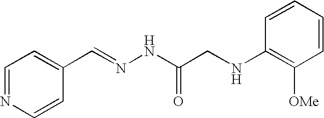

(2-Methoxy-phenylamino)-acetic acid [1-pyridin-4-yl-meth-(E)-ylidene]-hydrazide: In a similar fashion by making the appropriate substitution for the aniline in Example 1, Step 1, and substituting 4-pyridinecarboxaldehyde for 3,5-dichloro-4-pyridinecarboxaldehyde in Example 1, Step 3, the following compound was prepared: ¹H NMR (400 MHz, DMSO-d₆) δ ppm 3.79 (s, 3H) 4.25 (d, J=5.66 Hz, 2H) 5.16 (t, J=5.27 Hz, 1H) 6.52-6.62 (m, 2H) 6.73-6.80 (m, 1H) 6.82 (dd, J=7.91, 1.27 Hz, 1H) 7.65-7.70 (m, 2H) 7.98 (s, 1H) 8.58-8.65 (m, 2H) 11.84 (s, 1H); LCMS: 285 (M+H).

Results of the biochemical assay described above: GRK-2 +.

### Example 68

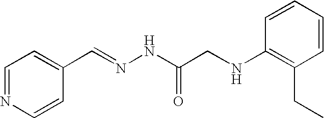

(2-Ethyl-phenylamino)-acetic acid [1-pyridin-4-yl-meth-(E)-ylidene]-hydrazide: In a similar fashion by making the appropriate substitution for the aniline in Example 1, Step 1, and substituting 4-pyridinecarboxaldehyde for 3,5-dichloro-4-pyridinecarboxaldehyde in Example 1, Step 3, the following compound was prepared: ¹H NMR (400 MHz, DMSO-d₆) δ ppm 1.16 (t, J=7.42 Hz, 3H) 2.44-2.56 (m, 2H) 4.29 (d, J=5.47 Hz, 2H) 5.08 (t, J=5.37 Hz, 1H) 6.49 (d, J=7.61 Hz, 1H) 6.53-6.61 (m, 1H) 6.95-7.03 (m, J=8.40, 8.40 Hz, 2H) 7.65-7.70 (m, 2H) 7.99 (s, 1H) 8.58-8.65 (m, 2H) 11.82 (s, 1H); LCMS: 283 (M+H).

Results of the biochemical assay described above: GRK-2 +.

### Example 69

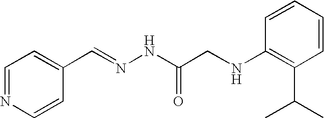

(2-Isopropyl-phenylamino)-acetic acid [1-pyridin-4-yl-meth-(E)-ylidene]-hydrazide: In a similar fashion by making the appropriate substitution for the aniline in Example 1, Step 1, and substituting 4-pyridinecarboxaldehyde for 3,5-dichloro-4-pyridinecarboxaldehyde in Example 1, Step 3, the following compound was prepared: ¹H NMR (400 MHz, DMSO-d₆) δ ppm 1.18 (d, J=6.83 Hz, 6H) 2.91-3.06 (m, J=6.83 Hz, 1H) 4.29 (t, J=5.17 Hz, 2H) 5.11-5.18 (m, 1H) 6.46-6.51 (m, 1H) 6.56-6.64 (m, 1H) 6.93-7.01 (m, 1H) 7.05 (dd, J=7.52, 1.27 Hz, 1H) 7.65-7.71 (m, 2H) 7.99 (s, 1H) 8.59-8.64 (m, 2H) 11.82 (s, 1H); LCMS: 297 (M+H).

Results of the biochemical assay described above: GRK-2 +.

### Example 70

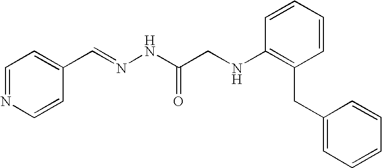

(2-Benzyl-phenylamino)-acetic acid [1-pyridin-4-yl-meth-(E)-ylidene]-hydrazide: In a similar fashion by making the appropriate substitution for the aniline in Example 1, Step 1, and substituting 4-pyridinecarboxaldehyde for 3,5-dichloro-4-pyridinecarboxaldehyde in Example 1, Step 3, the following compound was prepared: ¹H NMR (400 MHz, DMSO-d₆) δ ppm 3.85 (s, 2H) 4.25 (d, J=5.27 Hz, 2H) 5.13 (t, J=5.37 Hz, 1H) 6.51-6.60 (m, 2H) 6.92 (dd, J=7.32, 1.46 Hz, 1H) 6.99-7.08 (m, 1H) 7.12-7.32 (m, 5 H) 7.64-7.69 (m, 2H) 7.98 (s, 1H) 8.59-8.64 (m, 2H) 11.78 (s, 1H); LCMS: 345 (M+H).

Results of the biochemical assay described above: GRK-2 ++.

### Example 71

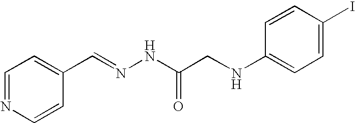

(4-Iodo-phenylamino)-acetic acid [1-pyridin-4-yl-meth-(E)-ylidene]-hydrazide: In a similar fashion by making the appropriate substitution for the aniline in Example 1, Step 1, and substituting 4-pyridinecarboxaldehyde for 3,5-dichloro-4-pyridinecarboxaldehyde in Example 1, Step 3, the following compound was prepared: ¹H NMR (400 MHz, DMSO-d₆) δ ppm 4.23 (d, J=5.86 Hz, 2H) 5.98-6.03 (m, 1H) 6.48 (d, J=8.79 Hz, 2H) 7.29-7.37 (m, 2H) 7.66 (dd, J=4.59, 1.46 Hz, 2H) 7.96 (s, 1H) 8.57-8.64 (m, 2H) 11.74 (s, 1H); LCMS: 381 (M+H).

Results of the biochemical assay described above: GRK-2 +.

### Example 72

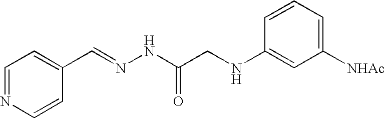

N-(3-{[1-Pyridin-4-yl-meth-(E)-ylidene-hydrazinocarbonylmethyl]-amino}-phenyl)-acetamide: In a similar fashion by making the appropriate substitution for the aniline in Example 1, Step 1, and substituting 4-pyridinecarboxaldehyde for 3,5-dichloro-4-pyridinecarboxaldehyde in Example 1, Step 3, the following compound was prepared: ¹H NMR (400 MHz, DMSO-d₆) δ ppm 1.97 (s, 3H) 4.20 (d, J=6.05 Hz, 2H) 5.74 (t, J=5.86 Hz, 1H) 6.27-6.33 (m, J=1.37 Hz, 1H) 6.73-7.07 (m, 4H) 7.62-7.67 (m, 2H) 7.97 (s, 2H) 9.63 (s, 1H) 11.71 (s, 1H); LCMS: 312 (M+H).

Results of the biochemical assay described above: GRK-2 +.

### Example 73

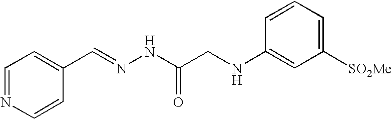

(3-Methanesulfonyl-phenylamino)-acetic acid [1-pyridin-4-yl-meth-(E)-ylidene]-hydrazide: In a similar fashion by making the appropriate substitution for the aniline in Example 1, Step 1, and substituting 4-pyridinecarboxaldehyde for 3,5-dichloro-4-pyridinecarboxaldehyde in Example 1, Step 3, the following compound was prepared: ¹H NMR (400 MHz, DMSO-d₆) δ ppm 3.10 (s, 3H) 4.34 (d, J=6.25 Hz, 2H) 6.41-6.47 (m, 1H) 6.88-6.93 (m, 1H) 7.06-7.12 (m, J=1.85, 1.85 Hz, 1H) 7.31 (t, J=7.91 Hz, 1H) 7.67 (dd, J=4.49, 1.56 Hz, 2H) 7.98 (s, 1H) 8.59-8.64 (m, 2H) 11.78 (s, 1H); LCMS: 333 (M+H).

Results of the biochemical assay described above: GRK-2 ++.

### Example 74

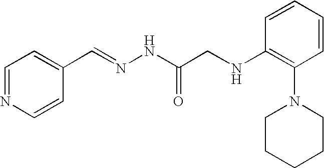

(2-Piperidin-1-yl-phenylamino)-acetic acid [1-pyridin-4-yl-meth-(E)-ylidene]-hydrazide: In a similar fashion by making the appropriate substitution for the aniline in Example 1, Step 1, and substituting 4-pyridinecarboxaldehyde for 3,5-dichloro-4-pyridinecarboxaldehyde in Example 1, Step 3, the following compound was prepared: ¹H NMR (400 MHz, DMSO-d₆) δ ppm 1.59-1.72 (m, 6H) 2.75 (s, 4H) 4.26 (d, J=5.47 Hz, 2H) 5.48 (t, J=5.47 Hz, 1H) 6.53-6.60 (m, 2H) 6.85-6.98 (m, 2H) 7.64-7.72 (m, 2H) 8.00 (s, 1H) 8.59-8.65 (m, 2H); LCMS: 338 (M+H).

Results of the biochemical assay described above: GRK-2 +.

### Example 75

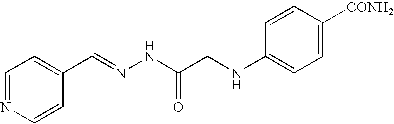

4-{[1-Pyridin-4-yl-meth-(E)-ylidene-hydrazinocarbonylmethyl]-amino}-benzamide: In a similar fashion by making the appropriate substitution for the aniline in Example 1, Step 1, and substituting 4-pyridinecarboxaldehyde for 3,5-dichloro-4-pyridinecarboxaldehyde in Example 1, Step 3, the following compound was prepared: ¹H NMR (400 MHz, DMSO-d₆) δ ppm 4.32 (t, J=5.08 Hz, 2H) 6.26-6.32 (m, 1H) 6.60 (d, J=8.79 Hz, 2H) 7.50-7.57 (m, 1H) 7.58-7.71 (m, 4H) 7.98 (s, 1H) 8.59-8.64 (m, J=5.27 Hz, 2H) 11.78 (s, 1H); LCMS: 298 (M+H).

Results of the biochemical assay described above: GRK-2 +.

### Example 76

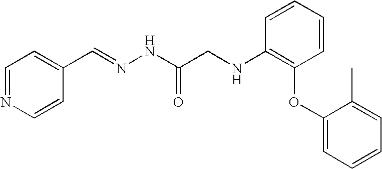

(3-o-Tolyloxy-phenylamino)-acetic acid [1-pyridin-4-yl-meth-(E)-ylidene]-hydrazide: In a similar fashion by making the appropriate substitution for the aniline in Example 1, Step 1, and substituting 4-pyridinecarboxaldehyde for 3,5-dichloro-4-pyridinecarboxaldehyde in Example 1, Step 3, the following compound was prepared: ¹H NMR (400 MHz, DMSO-d₆) δ ppm 2.23-2.24 (m, 3H) 4.28-4.34 (m, 2H) 5.32-5.38 (m, 1H) 6.52-6.64 (m, 2H) 6.69-6.77 (m, 1H) 6.90-7.05 (m, 2H) 7.09-7.17 (m, J=1.76 Hz, 1H) 7.23-7.28 (m, 1H) 7.64-7.70 (m, 2H) 7.97 (s, 1H) 8.59-8.64 (m, 2H) 11.81 (s, 1H); LCMS: 361 (M+H).

Results of the biochemical assay described above: GRK-2 +.

### Example 77

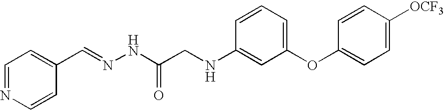

[3-(4-Trifluoromethoxy-phenoxy)-phenylamino]-acetic acid [1-pyridin-4-yl-meth-(E/Z)-ylidene]-hydrazide: In a similar fashion by making the appropriate substitution for the aniline in Example 1, Step 1, and substituting 4-pyridinecarboxaldehyde for 3,5-dichloro-4-pyridinecarboxaldehyde in Example 1, Step 3, the following compound was prepared: ¹H NMR (400 MHz, METHANOL-d₄) δ ppm 3.92 (s, 1H) 4.34 (s, 1H) 6.27-6.34 (m, 2H) 6.44-6.49 (m, 1H) 6.98-7.03 (m, 2H) 7.12 (dt, J=10.15, 8.10 Hz, 1H) 7.16-7.21 (m, 2H) 7.66-7.70 (m, 1H) 7.73-7.78 (m, 1H) 7.91 (s, 1H) 8.15 (s, 1H) 8.52-8.61 (m, 2H); LCMS: 431 (M+H).

Results of the biochemical assay described above: GRK-2 +++.

### Example 78

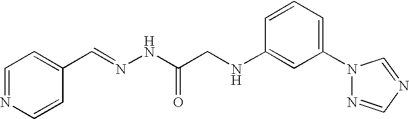

(3-[1,2,4]Triazol-1-yl-phenylamino)-acetic acid [1-pyridin-4-yl-meth-(E)-ylidene]-hydrazide: In a similar fashion by making the appropriate substitution for the aniline in Example 1, Step 1, and substituting 4-pyridinecarboxaldehyde for 3,5-dichloro-4-pyridinecarboxaldehyde in Example 1, Step 3, the following compound was prepared: ¹H NMR (400 MHz, DMSO-d₆) δ ppm 4.34 (d, J=5.66 Hz, 2H) 6.17-6.23 (m, 1H) 6.96-7.05 (m, 2H) 7.07 (t, J=2.05 Hz, 1H) 7.21 (t, J=8.00 Hz, 1H) 7.68 (d, J=5.86 Hz, 2H) 7.98 (s, 1H) 8.13 (s, 1H) 8.62 (s, 1H) 9.15 (s, 1H) 11.77 (s, 1H); LCMS: 322 (M+H).

Results of the biochemical assay described above: GRK-2 +.

### Example 79

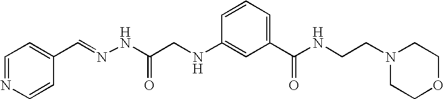

N-(2-Morpholin-4-yl-ethyl)-3-{[1-pyridin-4-yl-meth-(E)-ylidene-hydrazinocarbonylmethyl]-amino}-benzamide: In a similar fashion by making the appropriate substitution for the aniline in Example 1, Step 1, and substituting 4-pyridinecarboxaldehyde for 3,5-dichloro-4-pyridinecarboxaldehyde in Example 1, Step 3, the following compound was prepared: ¹H NMR (400 MHz, DMSO-d₆) δ ppm 2.34-2.43 (m, 8H) 3.50-3.55 (m, 6H) 4.27-4.31 (m, 2H) 5.91-5.96 (m, 1H) 6.73-6.77 (m, 1H) 6.95-7.02 (m, 2H) 7.08-7.15 (m, 1H) 7.66 (d, J=5.86 Hz, 2H) 7.97 (s, 1H) 8.11-8.17 (m, 1H) 8.58-8.63 (m, 2H); LCMS: 411 (M+H).

Results of the biochemical assay described above: GRK-2 +++.

### Example 80

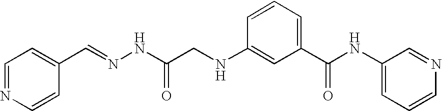

N-Pyridin-3-yl-3-{[1-pyridin-4-yl-meth-(E)-ylidene-hydrazinocarbonylmethyl]-amino}-benzamide: In a similar fashion by making the appropriate substitution for the aniline in Example 1, Step 1, and substituting 4-pyridinecarboxaldehyde for 3,5-dichloro-4-pyridinecarboxaldehyde in Example 1, Step 3,the following compound was prepared: ¹H NMR (400 MHz, DMSO-d₆) δ ppm 4.30-4.35 (m, 2H) 6.07 (s, 1H) 6.82-6.87 (m, 1H) 7.10-7.17 (m, 3H) 7.22 (t, J=7.91 Hz, 1H) 7.31-7.38 (m, 1H) 7.67 (dd, J=4.49, 1.56 Hz, 2H) 7.98 (s, 1H) 8.11-8.17 (m, 1H) 8.26 (dd, J=4.69, 1.37 Hz, 1H) 8.57-8.64 (m, 2H) 8.88 (d, J=2.34 Hz, 1H) 10.26 (s, 1H); LCMS: 375 (M+H).

Results of the biochemical assay described above: GRK-2 +++.

### Example 81

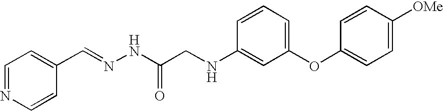

3-(4-Methoxy-phenoxy)-phenylamino]-acetic acid [1-pyridin-4-yl-meth-(E/Z)-ylidene]-hydrazide: In a similar fashion by making the appropriate substitution for the aniline in Example 1, Step 1, and substituting 4-pyridinecarboxaldehyde for 3,5-dichloro-4-pyridinecarboxaldehyde in Example 1, Step 3, the following compound was prepared: ¹H NMR (400 MHz, METHANOL-d₄) δ ppm 3.90-3.94 (m, 3H) 4.34 (s, 2H) 6.26-6.35 (m, 2H) 6.43-6.50 (m, 1H) 6.98-7.03 (m, 2H) 7.08-7.16 (m, 2H) 7.16-7.21 (m, 2H) 7.74-7.77 (m, 2H) 8.15 (s, 1H) 8.55-8.59 (m, 2H); LCMS: 377 (M+H).

Results of the biochemical assay described above: GRK-2 ++++.

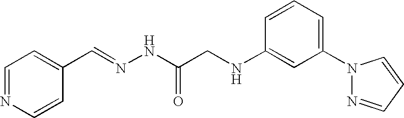

### Example 82

(3-Pyrazol-1-yl-phenylamino)-acetic acid [1-pyridin-4-yl-meth-(E)-ylidene]-hydrazide: In a similar fashion by making the appropriate substitution for the aniline in Example 1, Step 1, and substituting 4-pyridinecarboxaldehyde for 3,5-dichloro-4-pyridinecarboxaldehyde in Example 1, Step 3, the following compound was prepared: ¹H NMR (400 MHz, METHANOL-d₄) δ ppm 4.02 (s, 1H) 4.43 (s, 1H) 6.42-6.50 (m, 1H) 6.57-6.69 (m, 1H) 6.90-7.06 (m, 2H) 7.15-7.28 (m, 1H) 7.60-7.80 (m, 3H) 7.83-7.88 (m, 1H) 7.94 (s, 1H) 8.10 (d, J=2.54 Hz, 1H) 8.17 (s, 1H) 8.49-8.63 (m, 2H) 8.65-8.70 (m, 1H); LCMS: 321 (M+H).

Results of the biochemical assay described above: GRK-2 +.

### Example 83

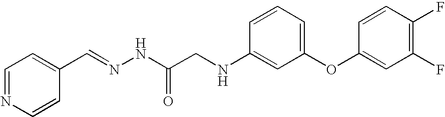

[3-(3,4-Difluoro-phenoxy)-phenylamino]-acetic acid [1-pyridin-4-yl-meth-(E)-ylidene]-hydrazide: In a similar fashion by making the appropriate substitution for the aniline in Example 1, Step 1, and substituting 4-pyridinecarboxaldehyde for 3,5-dichloro-4-pyridinecarboxaldehyde in Example 1, Step 3, the following compound was prepared: ¹H NMR (400 MHz, METHANOL-d₄) δ ppm 3.92 (s, 1H) 4.34 (s, 1H) 6.23-6.36 (m, 2H) 6.42-6.52 (m, 1H) 6.70-6.78 (m, 1H) 6.81-6.90 (m, 1H) 7.02-7.23 (m, 3H) 7.65-7.70 (m, 1H) 7.73-7.78 (m, 1H) 7.91 (s, 1H) 8.15 (s, 1H) 8.52-8.63 (m, 2H); LCMS: 383 (M+H).

Results of the biochemical assay described above: GRK-2 +++.

### Example 84

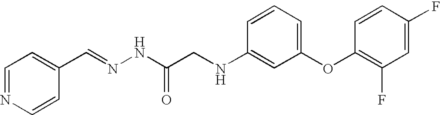

[3-(2,4-Difluoro-phenoxy)-phenylamino]-acetic acid [1-pyridin-4-yl-meth-(E)-ylidene]-hydrazide: In a similar fashion by making the appropriate substitution for the aniline in Example 1, Step 1, and substituting 4-pyridinecarboxaldehyde for 3,5-dichloro-4-pyridinecarboxaldehyde in Example 1, Step 3, the following compound was prepared: ¹H NMR (400 MHz, METHANOL-d₄) δ ppm 3.90 (s, 1H) 4.31 (s, 1H) 6.15-6.25 (m, 2H) 6.34-6.45-(m, 1H) 6.84-6.94 (m, 1H) 6.97-7.15 (m, 4H) 7.65-7.70 (m, 1H) 7.72-7.79 (m, 1H) 7.91 (s, 1H) 8.15 (s, 1H) 8.50-8.64 (m, 2H); LCMS: 383 (M+H).

Results of the biochemical assay described above: GRK-2 ++++.

### Example 85

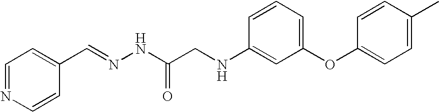

(3-p-Tolyloxy-phenylamino)-acetic acid [1-pyridin-4-yl-meth-(E)-ylidene]-hydrazide: In a similar fashion by making the appropriate substitution for the aniline in Example 1, Step 1, and substituting 4-pyridinecarboxaldehyde for 3,5-dichloro-4-pyridinecarboxaldehyde in Example 1 Step 3 the following compound was prepared: ¹H NMR (400 MHz, DMSO-d₆) δ ppm 2.22 (s, 3H) 4.17-4.23 (m, 2H) 5.91-6.39 (m, 4H) 6.85 (d, J=8.20 Hz, 2H) 6.94-7.07 (m, 1H) 7.06 -7.18 (m, 1H) 7.55-7.67 (m, J=6.05 Hz, 2H) 7.94 (s, 1H) 8.20 (s, 1H) 8.61 (s, 2H); LCMS: 361 (M+H).

Results of the biochemical assay described above: GRK-2 ++++.

### Example 86

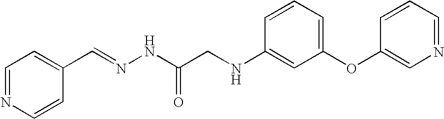

[3-(Pyridin-3-yloxy)-phenylamino]-acetic acid [1-pyridin-4-yl-meth-(E)-ylidene]-hydrazide: In a similar fashion by making the appropriate substitution for the aniline in Example 1, Step 1, and substituting 4-pyridinecarboxaldehyde for 3,5-dichloro-4-pyridinecarboxaldehyde in Example 1, Step 3, the following compound was prepared: ¹H NMR (400 MHz, DMSO-d₆) δ ppm 3.80 (d, J=6.64 Hz, 2H) 4.22 (d, J=5.86 Hz, 2H) 6.04 (t, J=6.35 Hz, 1H) 6.16-6.23 (m, 2H) 6.25-6.28 (m, 1H) 6.32 (t, J=2.05 Hz, 1H) 6.36-6.48 (m, 2H) 7.03-7.14 (m, 2H) 7.56-7.67 (m, 3H) 7.94 (s, 1H) 8.19 (s, 1H) 8.25-8.34 (m, 3H) 8.57-8.65 (m, 3H) 11.72 (s, 1H); LCMS: 348 (M+H).

Results of the biochemical assay described above: GRK-2 +++.

### Example 87

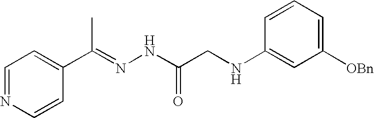

(3-Benzyloxy-phenylamino)-acetic acid [1-pyridin-4-yl-eth-(E)-ylidene]-hydrazide: In a similar fashion by making the appropriate substitution for the aniline in Example 1, Step 1, and substituting 4-acetylpyridine for 3,5-dichloro-4-pyridinecarboxaldehyde in Example 1, Step 3 the following compound was prepared: ¹H NMR (400 MHz, DMSO-d₆) δ ppm 2.24 (s, 3H) 4.25 (d, J=5.47 Hz, 2H) 4.98 (s, 2H) 5.71-5.80 (m, 1H) 6.16-6.27 (m, 3H) 6.90-7.00 (m, 1H) 7.23-7.43 (m, 5 H) 7.74 (s, 2H) 8.60 (d, J=6.05 Hz, 2H) 10.93 (s, 1H); LCMS: 375 (M+H).

Results of the biochemical assay described above:

| | |
|---|---|
| GRK-2 | +++ |
| GRK-3 | +++ |
| GRK-5 | + |
| GRK-6 | −−. |

### Example 88

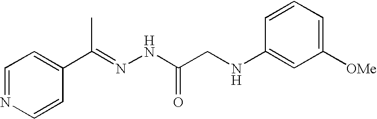

(3-Methoxy-phenylamino)-acetic acid [1-pyridin-4-yl-eth-(E)-ylidene]-hydrazide: In a similar fashion by making the appropriate substitution for the aniline in Example 1, Step 1 and substituting 4-acetylpyridine for 3,5-dichloro-4-pyridinecarboxaldehyde in Example 1, Step 3 the following compound was prepared: ¹H NMR (400 MHz, DMSO-d₆) δ ppm 2.23 (s, 3H) 3.62 (s, 3H) 3.84-3.92 (m, 1H) 4.19-4.27 (m, 1H) 5.73 (s, 1H) 6.18 (d, J=15.81 Hz, 3H) 6.94 (s, 1H) 7.72 (s, 2H) 8.58 (s, 2H); LCMS: 299 (M+H).

Results of the biochemical assay described above:

| | |
|---|---|
| GRK-2 | +++ |
| GRK-3 | +++ |
| GRK-5 | ++ |
| GRK-6 | +. |

### Example 89

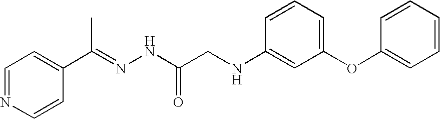

(3-Phenoxy-phenylamino)-acetic acid [1-pyridin-4-yl-eth-(E)-ylidene]-hydrazide: In a similar fashion by making the appropriate substitution for the aniline in Example 1, Step 1 and substituting 4-acetylpyridine for 3,5-dichloro-4-pyridinecarboxaldehyde in Example 1, step 3 the following compound was prepared: ¹H NMR (400 MHz, DMSO-d₆) δ ppm 2.21 (s, 3H) 4.19-4.27 (m, 2H) 5.92-6.01 (m, 1H) 6.09-6.19 (m, 1H) 6.25 (t, J=2.15 Hz, 1H) 6.38 (dd, J=8.20, 1.56 Hz, 1H) 6.90-6.98 (m, 2H) 6.99-7.08 (m, 2H) 7.26-7.36 (m, 2H) 7.69 (d, J=5.86 Hz, 2H) 10.91 (s, 1H); LCMS: 361 (M+H).

Results of the biochemical assay described above:

| | |
|---|---|
| GRK-2 | +++ |
| GRK-3 | +++ |
| GRK-5 | + |
| GRK-6 | −−. |

### Example 90

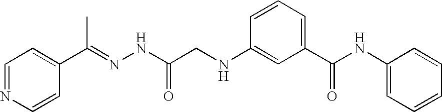

N-Phenyl-3-{[1-pyridin-4-yl-eth-(E)-ylidene-hydrazinocarbonylmethyl]-amino}-benzamide: In a similar fashion by making the appropriate substitution for the aniline in Example 1, Step 1 and substituting 4-acetylpyridine for 3,5-dichloro-4-pyridinecarboxaldehyde in Example 1, Step 3 the following compound was prepared: ¹H NMR (400 MHz, METHANOL-d₄) δ ppm 4.10 (s, 3H) 4.47 (s, 4H) 6.88 (d, J=8.00 Hz, 4H) 7.12 (t, J=7.42 Hz, 3H) 7.16-7.29 (m, 10 H) 7.32 (t, J=7.91 Hz, 7H) 7.64 (d, J=7.81 Hz, 7H) 7.85 (dd, J=10.35, 5.86 Hz, 7H) 8.55 (dd, J=9.47, 5.76 Hz, 7H); LCMS: 388 (M+H).

Results of the biochemical assay described above:

| | |
|---|---|
| GRK-2 | +++ |
| GRK-3 | +++ |
| GRK-5 | + |
| GRK-6 | −−. |

### Example 91

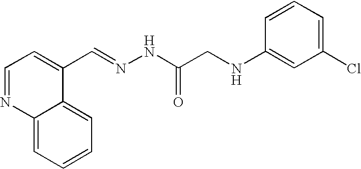

(3-Chloro-phenylamino)-acetic acid [1-quinolin-4-yl-meth-(E)-ylidene]-hydrazide: In a similar fashion by making the appropriate substitution for the aniline in Example 1, Step 1 and substituting 4-quinolinecarboxaldehyde for 3,5-dichloro-4-pyridinecarboxaldehyde in Example 1, Step 3 the following compound was prepared: ¹H NMR (400 MHz, DMSO-d₆) δ ppm 4.17 (s, 2H) 5.92 (s, 1H) 6.60 (s, 2H) 6.66-6.71 (m, 1H) 7.04-7.11 (m, 1H) 7.64-7.72 (m, 1H) 7.75-7.83 (m, 2H) 8.04-8.11 (m, 1H) 8.56-8.64 (m, 1H) 8.78 (s, 1H) 8.94 (t, J=4.78 Hz, 1H) 11.46 (s, 1H); LCMS: 339 (M+H).

Results of the biochemical assay described above: GRK-2 +.

### Example 92

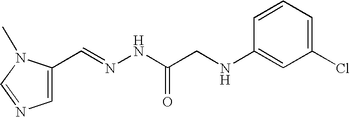

(3-Chloro-phenylamino)-acetic acid [1-(3-methyl-3H-imidazol-4-yl)-meth-(E)-ylidene]-hydrazide: In a similar fashion by making the appropriate substitution for the aniline in Example 1, Step 1 and substituting 1-methyl-2-imidazolecarboxyldehyde for 3,5-dichloro-4-pyridinecarboxaldehyde in Example 1, Step 3 the following compound was prepared: ¹H NMR (400 MHz, DMSO-d₆) δ ppm 3.85 (s, 3H) 4.15 (d, J=5.86 Hz, 2H) 6.09 (t, J=5.86 Hz, 1H) 6.48-6.62 (m, 3H) 7.01-7.09 (m, 1H) 7.29 (d, J=0.98 Hz, 1H) 7.75 (s, 1H) 7.96 (s, 1H) 11.35 (s, 1H); LCMS: 292 (M+H).

Results of the biochemical assay described above: GRK-2 +.

### Example 93

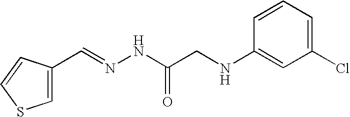

(3-Chloro-phenylamino)-acetic acid [1-thiophen-3-yl-meth-(E)-ylidene]-hydrazide: In a similar fashion by making the appropriate substitution for the aniline in Example 1, Step 1 and substituting 3-thiophenecarboxaldehyde for 3,5-dichloro-4-pyridinecarboxaldehyde in Example 1, step 3 the following compound was prepared: ¹H NMR (400 MHz, DMSO-d₆) δ ppm 4.26 (d, J=5.86 Hz, 2H) 6.17 (t, J=5.86 Hz, 1H) 6.57-6.68 (m, 3H) 6.71 (t, J=2.05 Hz, 1H) 7.13 (t, J=8.00 Hz, 1H) 7.60 (dd, J=5.08, 0.98 Hz, 1H) 7.65-7.71 (m, 1H) 7.94-7.98 (m, 1H) 8.11 (s, 1H) 11.47 (s, 1H); LCMS: 294 (M+H).

Results of the biochemical assay described above: GRK-2 +.

### Example 94

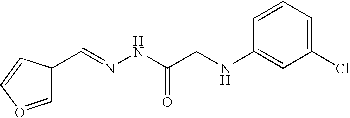

(3-Chloro-phenylamino)-acetic acid [1-furan-3-yl-meth-(E)-ylidene]-hydrazide: In a similar fashion by making the appropriate substitution for the aniline in Example 1, Step 1 and substituting 3-furancarboxaldehyde for 3,5-dichloro-4-pyridinecarboxaldehyde in Example 1, step 3 the following compound was prepared: ¹H NMR (400 MHz, DMSO-d₆) δ ppm 4.23 (d, J=5.86 Hz, 2H) 6.16 (t, J=5.86 Hz, 1H) 6.57-6.67 (m, 2H) 6.70 (t, J=2.05 Hz, 1H) 6.94 (d, J=1.76 Hz, 1H) 7.13 (t, J=8.10 Hz, 1H) 7.78-7.83 (m, 1H) 8.02 (s, 1H) 8.18 (s, 1H) 11.46 (s, 1H); LCMS: 278 (M+H).

Results of the biochemical assay described above: GRK-2 ++.

### Example 95

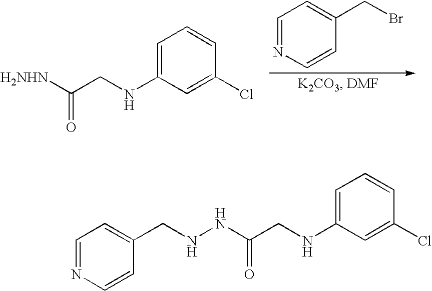

(3-Chloro-phenylamino)-acetic acid N′-pyridin-4-ylmethyl-hydrazide: A 10-mL round bottom flask equipped with a stirrer bar was charged with (3-Chloro-phenylamino)-acetic acid hydrazide (200 mg, 1.0 mmol, see Example 1, Step 2), potassium carbonate (692 mg, 5.0 mmol), and 4-bromomethylpyridne (228 mg, 0.9 mmol) in DMF (4.0 mL) and stirred at 25° C. for 24 h. The reaction was filtered and concentrated in vacuo and purified by prep-HPLC to afford product (20 g, 8%) as an oil. ¹H NMR (400 MHz, CHLOROFORM-d) δ ppm 3.48 (d, J=2.93 Hz, 2H) 3.81 (d, J=5.66 Hz, 2H) 4.00 (d, J=4.49 Hz, 2H) 4.18-4.25 (m, J=4.69 Hz, 1H) 6.37-6.43 (m, 1H) 6.53 (t, J=2.15 Hz, 1H) 6.75-6.80 (m, 1H) 7.07 (t, J=8.10 Hz, 1H) 7.20 (d, J=6.05 Hz, 2H) 8.48-8.52 (m, 2H); LCMS: 291 (M+H).

Results of the biochemical assay described above: GRK-2 +.

### Example 96

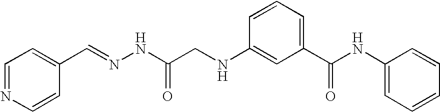

N-Phenyl-3-{[1-pyridin-4-yl-meth-(E)-ylidene-hydrazinocarbonylmethyl]-amino}-benzamide: In a similar fashion by making the appropriate substitution for the aniline in Example 1, Step 1, and substituting 4-pyridinecarboxaldehyde for 3,5-dichloro-4-pyridinecarboxaldehyde in Example 1, Step 3, the following compound was prepared: ¹H NMR (400 MHz, DMSO-d₆) δ ppm 4.31-4.36 (m, 2H) 6.01-6.07 (m, 1H) 6.83 (d, J=8.00 Hz, 1H) 7.05 (t, J=7.52 Hz, 1H) 7.08-7.14 (m, 2H) 7.20 (t, J=7.91 Hz, 1H) 7.30 (t, J=8.00 Hz, 2H) 7.67 (d, J=6.05 Hz, 1H) 7.70-7.75 (m, 2H) 7.98 (s, 1H) 8.58-8.63 (m, 2H) 10.05 (s, 2H); LCMS: 361 (M+H).

Results of the biochemical assay described above:

| | |
|---|---|
| GRK-2 | +++ |
| GRK-3 | ++++ |
| GRK-5 | + |
| GRK-6 | −−. |

### Example 97

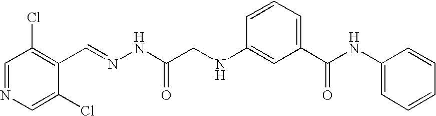

3-{[1-(3,5-Dichloro-pyridin-4-yl)-meth-(E)-ylidene-hydrazinocarbonylmethyl]-amino}-N-phenyl-benzamide: In a similar fashion by making the appropriate substitution for the aniline the following compound was prepared according to Example 1: ¹H NMR (400 MHz, DMSO-d₆) δ ppm 4.28 (d, J=5.86 Hz, 2H) 6.78 (d, J=9.57 Hz, 1H) 7.02-7.09 (m, 2H) 7.11 (d, J=6.25 Hz, 1H) 7.19 (t, J=7.81 Hz, 1H) 7.30 (t, J=7.91 Hz, 2H) 7.72 (d, J=7.42 Hz, 2H) 8.68 (s, 2H) 11.97 (s, 1H); LCMS: 441 (M+H).

Results of the biochemical assay described above:

| | |
|---|---|
| GRK-2 | +++ |
| GRK-3 | ++++ |
| GRK-5 | ++ |
| GRK-6 | +. |

### Example 98

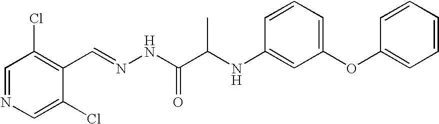

2-(3-Phenoxy-phenylamino)-propionic acid [1-(3,5-dichloro-pyridin-4-yl)-meth-(E)-ylidene]-hydrazide: In a similar fashion by making the appropriate substitution for the aniline and substituting 2-bromo-ethylpropionate for bromo-ethylacetate the following compound was prepared according to Example 1: ¹H NMR (400 MHz, DMSO-d₆) δ ppm 1.31 (d, J=6.83 Hz, 3H) 3.37-3.46 (m, 1H) 4.31 (t, J=4.98 Hz, 1H) 6.08-6.20 (m, 2H) 6.23-6.38 (m, 1H) 6.86-7.10 (m, 4H) 7.21-7.28 (m, 2H) 8.24 (s, 1H) 8.67 (s, 2H) 11.85 (s, 1H); LCMS: 429 (M+H).

Results of the biochemical assay described above: GRK-2 +.

### Example 99

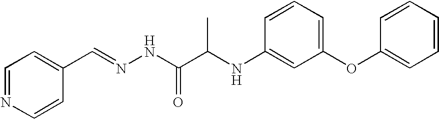

2-(3-phenoxy-phenylamino)-propionic acid [1-pyridin-4-yl-meth-(E)-ylidene]-hydrazide: In a similar fashion by making the appropriate substitution for the aniline and substituting 2-bromo-ethylpropionate for bromo-ethylacetate in Example 1, Step 1, and substituting 4-pyridinecarboxyaldehyde for 3,5-dichloro-4-pyridinecarboxyaldehyde in Example 1, Step 3, the following compound was prepared: ¹H NMR (400 MHz, DMSO-d₆) δ ppm 1.31-1.36 (m, 3H) 3.89-3.95 (m, 1H) 4.82-4.88 (m, 1H) 6.06-6.18 (m, 2H) 6.24 (t, J=2.15 Hz, 1H) 6.33 (t, J=7.22 Hz, 1H) 7.22-7.33 (m, 2H) 7.56-7.60 (m, 2H) 8.20 (s, 1H) 8.57-8.62 (m, 2H) 11.72 (s, 1H); LCMS: 361 (M+H).

Results of the biochemical assay described above: GRK-2 +.

### Example 100

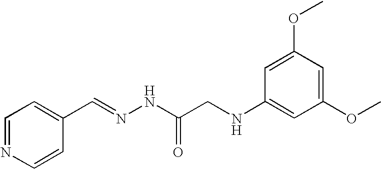

(3,5-Dimethoxy-phenylamino)-acetic acid [1-pyridin-4-yl-meth-(E)-ylidene]-hydrazide: In a similar fashion by making the appropriate substitution for the aniline in Example 1, Step 1, and substituting 4-pyridinecarboxaldehyde for 3,5-dichloro-4-pyridinecarboxaldehyde in Example 1, Step 3, the following compound was prepared: LCMS: 315 (M+H).

Results of the biochemical assay described above: GRK-2 +.

### Example 101

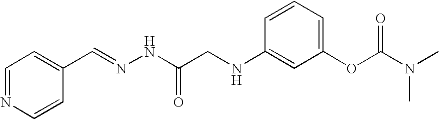

Dimethyl-carbamic acid 3-{[1-pyridin-4-yl-meth-(E)-ylidene-hydrazinocarbonyl-methyl]-amino}-phenyl ester: In a similar fashion by making the appropriate substitution for the aniline in Example 1, Step 1, and substituting 4-pyridinecarboxaldehyde for 3,5-dichloro-4-pyridinecarboxaldehyde in Example 1, Step 3, the following compound was prepared: LCMS: 429 (M+H).

Results of the biochemical assay described above: GRK-2 +.

### Reference Example Two

Cell-based porcine trabecular meshwork (PTM) assay.

The anterior section of porcine eyes was harvested within 4 hours post-mortem. The iris and ciliary body were removed and trabecular meshwork cells were harvested by blunt dissection. Finely minced trabecular meshwork tissue was plated into collagen-coated 6-well plates in Medium-199 containing 20% fetal bovine serum (FBS). After two passages at confluence, cells were transferred to low-glucose DMEM containing 10% FBS. Cells were used between passage 3 and passage.

Cells were plated into fibronectin-coated, glass multiwell plates the day before compound testing under standard culture conditions. Compounds were added to cells in the presence of 1% FBS-containing DMEM and 1% DMSO. When compounds were incubated with the cells for the duration determined to be optimal, the media and compound is removed and cells fixed for 20 minutes in 3% methanol-free paraformaldehyde. Cells were rinsed twice with phosphate buffered saline (PBS) and cells are permeabilized with 0.5% Triton X-100 for two minutes. Following an additional two washes with PBS, F-actin was stained with Alexa-fluor 488-labelled phalloidin and nuclei are stained with DAPI.

Data was reduced to the mean straight actin-fiber length and normalized to DMSO-treated control cells (100%) and 50 μM Y-27632 (0%). Y-27632 is a rho-kinase inhibitor known to result in the depolymerization of F-actin in these cells.

### Example 102

The cellular assay described in Reference Example Two was used to test some of the foregoing Examples 1-101, the results of which are presented below. The results are provided in terms activity at 50 μM as compared to control

| | |
|---|---|
| More active than control- | +++ |
| As active as control | ++ |
| Less active than control | + |
| Inactive | − |

TABLE 1

| Compound | PTM Cell Assay Result |
|---|---|
| 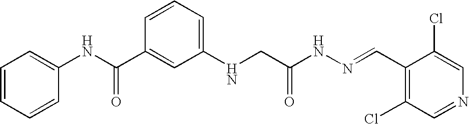 | ++ |
| 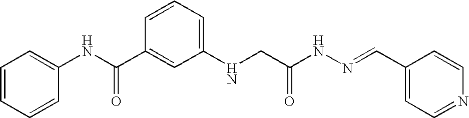 | ++ |
| 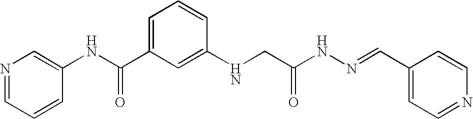 | ++ |
| 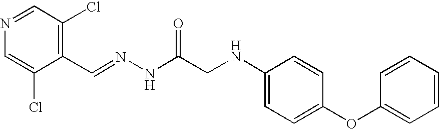 | + |
| 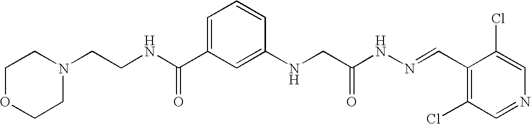 | + |
| 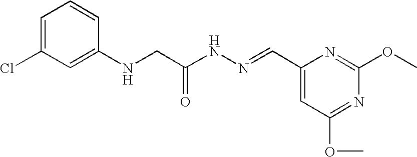 | + |
| 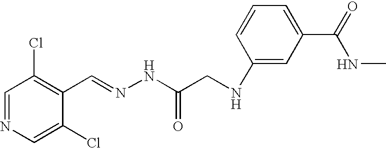 | + |
| 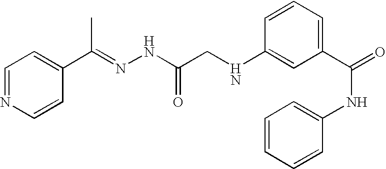 | + |
| 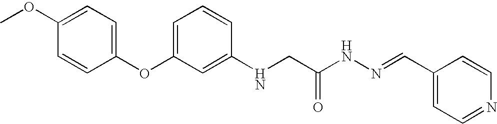 | + |

### Reference Example Three

Pharmacological Activity for Glaucoma Assay.

Pharmacological activity for glaucoma can be demonstrated using assays designed to test the ability of the subject compounds to decrease intraocular pressure. Examples of such assays are described in the following reference, incorporated herein: C. Liljebris, G. Selen, B. Resul, J. Sternschantz, and U. Hacksell, “Derivatives of 17-Phenyl-18,19,20-trinorprostaglandin F₂_{^{α}} Isopropyl Ester: Potential Antiglaucoma Agents”, Journal of Medicinal Chemistry, Vol. 38 (2) 1995, pp. 289-304.

### Example 103

Topical pharmaceutical compositions for lowering intraocular pressure are prepared by conventional methods and formulated as follows:

| Ingredient | Amount (wt %) |
|---|---|
| Hydrazide Derivative | 0.50 |
| Dextran 70 | 0.1 |
| Hydroxypropyl methylcellulose | 0.3 |
| Sodium Chloride | 0.77 |
| Potassium chloride | 0.12 |
| Disodium EDTA | 0.05 |
| Benzalkonium chloride | 0.01 |
| HCl and/or NaOH | pH 7.0-7.2 |
| Purified water | q.s. to 100% |

A compound according to this invention is used as the hydrazide derivative. When the composition is topically administered to the eyes once daily, the above composition decreases intraocular pressure in a patient suffering from glaucoma.

### Example 104

Example 103 is repeated using N-(2-Morpholin-4-yl-ethyl)-3-{[1-pyridin-4-yl-meth-(E)-ylidene-hydrazino-carbonylmethyl]-amino}-benzamide according to this invention. When administered as a drop 4 times per day, the above composition substantially decreases intraocular pressure and serves as a neuroprotective agent.

### Example 105

Example 103 is repeated using 4-{[1-(3,5-dichloro-pyridin-4-yl)-meth-(E)-ylidene-hydrazinocarbonylmethyl]-amino}-benzamide: according to this invention. When administered as a drop twice per day, the above composition substantially decreases intraocular pressure.

### Example 106

Example 103 is repeated using (3-p-Tolyloxy-phenylamino)-acetic acid [1-pyridin-4-yl-meth-(E)-ylidene]-hydrazide according to this invention. When administered as a drop twice per day, the above composition substantially decreases allergic symptoms and relieves dry eye syndrome.

### Example 107

Example 103 is repeated using a 3-{[1-Pyridin-4-yl-meth-(E)-ylidene-hydrazinocarbonylmethyl]-amino}-benzamide according to this invention. When administered as a drop as needed, the above composition substantially decreases hyperemia, redness and ocular irritation.

### Example 108

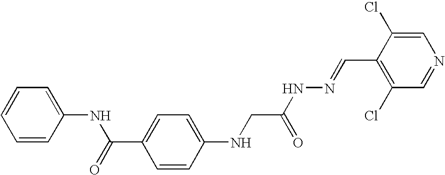

(E) 4-(2-(2-((3,5-dichloropyridin-4-yl)methylene)hydrazinyl)-2-oxoethylamino)-N-phenylbenzamide In a similar fashion by making the appropriate substitutions in Example 1, the title compound is prepared.

### Example 109

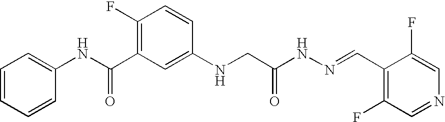

### (E)-5-(2-(2-((3,5-difluoropyridin-4-yl)methylene)hydrazinyl)-2-oxoethylamino)-2-fluoro-N-phenylbenzamide

In a similar fashion by making the appropriate substitution for the aniline in Example 1, Step 1, and substituting the appropriate compounds in Example 1, the title compound is prepared.

### Example 110

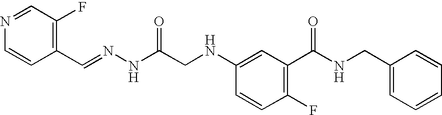

### (E)-N-benzyl-2-fluoro-5-(2-(2-((3-fluoropyridin-4-yl)methylene)hydrazinyl)-2-oxoethylamino)benzamide

In a similar fashion by making the appropriate substitution for the aniline in Example 1, Step 1, and substituting the appropriate compounds in Example 1, the title compound is prepared.

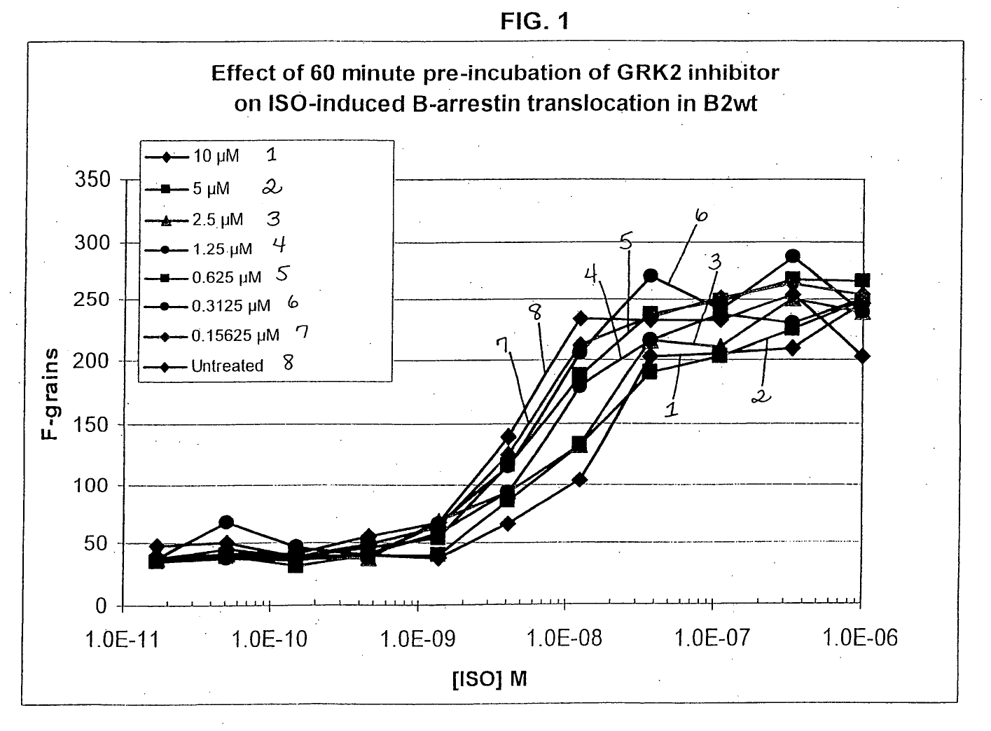

FIGS. 1-3 show the effect of the hydrazide of Example 41 in the translocation of several receptors. The Transfluor™ assay (Assay and Drug Development Technologies, Volume 1, Number 1-1, pages 21-30 (2002); U.S. Pat. Nos 5,891,646, and 6,110,693, each incorporated herein by reference in its entirety) was used to measure the degree of translocation in U2OS cells that over express the receptor and arrestin.

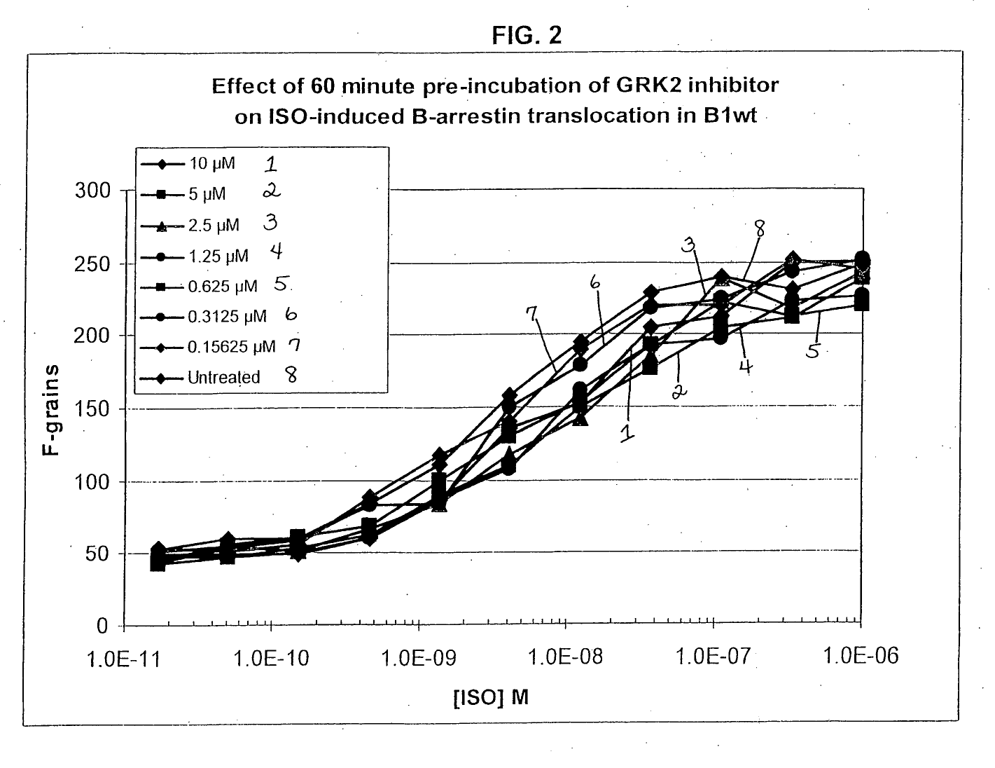

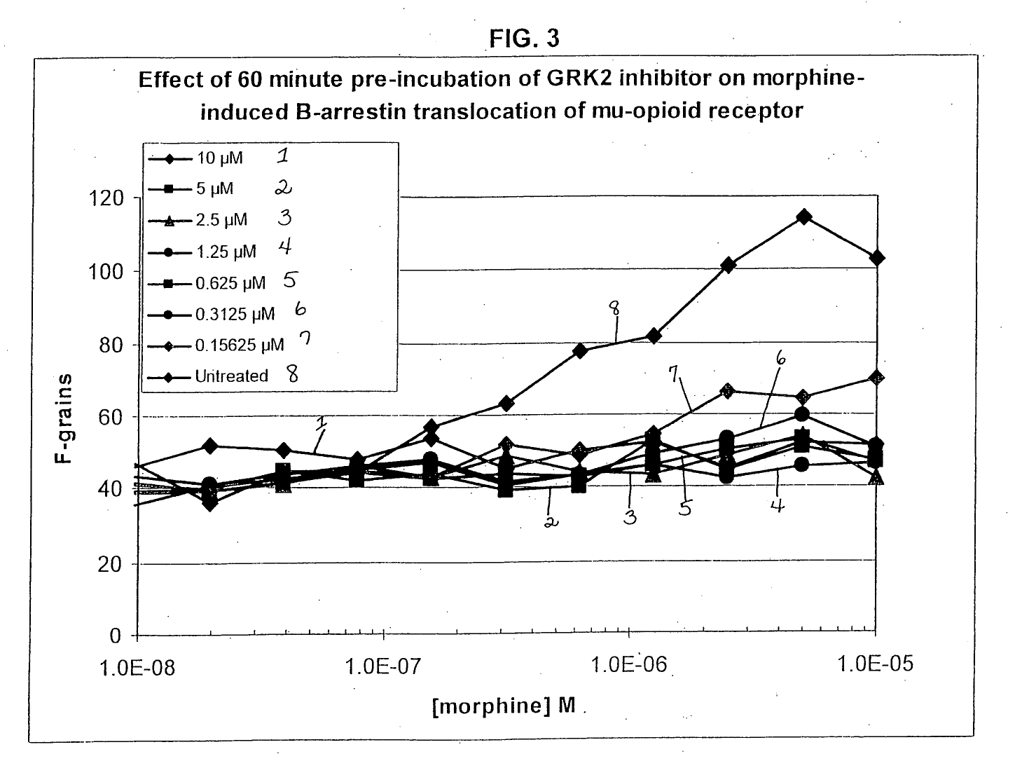

FIG. 1 shows the effect of the hydrazide of Example 41, in the isoproterenol-induced translocation of arrestin-GFP to the Beta 2 Adrenergic Receptor (B2 wt). FIG. 1 shows dose response curves for the effect of isoproterenol (a Beta 2 Adrenergic receptor agonist) against F-grains (a measure of the degree of arrestin-GFP translocation to the receptor) for increasing concentrations of the hydrazide of example 41 (see curves). For any given concentration of isoproterenol, as the concentration of the hydrazide increases, there is a stepwise reduction in translocation (decrease in F-grains). By preventing GRK-2 mediated phosphorylation of the receptor, the hydrazide of example 41 prevents binding of arrestin-GFP to the receptor. FIG. 2 shows the same effect on the Beta 1 Adrenergic receptor (B1 wt), and FIG. 3 is the effect on the mu opioid receptor.

The hydrazide of Example 41 is a very good inhibitor of GRK-2 and shows modest inhibition of arrestin-GFP translocation to the B2 WT and B1 WT, but strong inhibition to the μ opioid. Finally, increased concentrations of the hydrazide of Example 41 showed increased accumulation of cAMP in HEK-293 cells (Beta 2 Adrenergic receptor overexpressed) in the presence of a fixed concentration of isoproterenol. This is consistent with the inhibition of GRK-2 resulting in less translocation of arrestin-GFP, less desensitization, and consequently more signaling by the B2AR. With more receptors available now on the surface of the cell, more cAMP is being generated in the presence of isoproterenol.

In FIG. 1, the IC₅₀ of the β2 arrestin was 4 μM. In FIG. 2, the IC₅₀ of the β1 arrestin was greater than 10 μM. In FIG. 3, the IC₅₀ for the μ-opioid receptor was about 150 nM.

The hydrazides may further be screened for effect on GPCR desensitization by use of the methods described in U.S. Patent Application No. 2004/0091946, published May 13, 2004, or U.S. Patent Application No. 2005/0032125, published Feb. 10, 2005, both incorporated herein by reference in their entirety.

While the invention has been described in detail and with reference to specific embodiments thereof, it will be apparent to one skilled in the art that various changes and modifications can be made without departing from the spirit and scope of the invention. For example, any and all optical and other isomers are specifically contemplated.

What is claimed is:

1. A compound according to formula (I): 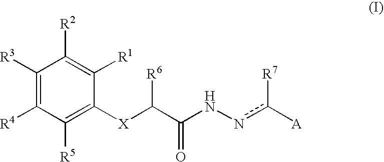
wherein [symbol]
may be a single or double bond;
A is selected from
a heteroaryl group (i): 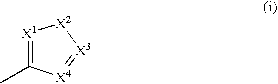
wherein X¹, X², X³ and X⁴ are, independently, CH, O, S or N—R⁶, with the proviso that at least one of X² or X³ is O, S or N—R⁶; and
a heteroaryl group (ii): 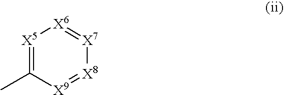
wherein X⁵and X⁹ are CH or C-halogen, X⁶ and X⁸ are CH, and X⁷ is N, and wherein the six-membered heteroaryl group may be further fused with an unsubstituted six-member aryl group;
R¹, R², R³, R⁴, and R⁵ are, independently, hydrogen; halogen; C₁-C₄ alkyl; amino; nitro; cyano; heteroaryl; carboxy, carbonylamino; aminosulfonyl; sulfonylamino; aminoacyl; thioalkyl; sulfonyl; acyl; heterocycle; —OR; —O—C₁-C₄alkyl-heterocycle; —C(O)NH—C₁-C₄alkyl-heterocycle; —C(O)NH-heteroaryl; —C(O)NH-aryl; or carboxylamino; wherein
R is C₁-C₄ alkyl; aryl, heteroaryl, C₁-C₄ alkyl aryl or C₁-C₄ alkyl heteroaryl;
R⁶ is H or C₁-C₄ alkyl;
R⁷is hydrogen, C₁-C₄ alkyl or C₁-C₄ alkoxy; and
X is N—R⁶.

2. A compound according to claim 1 wherein X is NH.

3. A compound according to claim 1 wherein each of R¹, R³ and R⁵ are hydrogen and at least one of R² or R⁴ is hydrogen.

4. A compound according to claim 3 wherein A is heteroaryl group (i).

5. A compound according to claim 3 wherein A is heteroaryl group (ii).

6. A compound according to claim 1 wherein [symbol]
is a double bond.

7. A compound according to claim 1 wherein R¹, R², R³, R⁴, and R⁵ are not carboxy.

8. A compound according to claim 1 wherein R¹, R², R³, R⁴, and R⁵ are, independently, carboxy, carbonylamino, sulfonylamino or acyl.

9. A compound according to claim 1 wherein R¹ is hydrogen and R² and R³ are independently selected from carboxy, carbonylamino, sulfonylamino or acyl.

10. A compound according to formula (II): 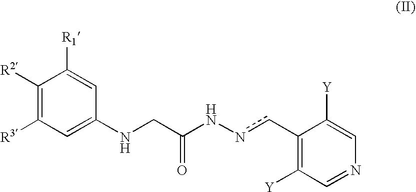
wherein [symbol]
may be a single or double bond;
R^{1′}, R^{2′} and R^{3′} are independently selected from hydrogen; halogen; C₁-C₄ alkyl; amino; nitro; cyano; heteroaryl; carboxyl, carbonylamino; aminosulfonyl; sulfonylamino; aminoacyl; thioalkyl; sulfonyl; acyl; heterocycle; —OR; —SR; —C(O)NH-heteroaryl; or —C(O)NH-aryl;
R is C₁-C₄ alkyl; aryl, heteroaryl, C₁-C₄ alkyl aryl or C₁-C₄ alkyl heteroaryl; and
Y is hydrogen, halogen or nitrile.

11. A compound according to claim 10 wherein Y is H or Cl.

12. A compound according to claim 11 wherein [symbol]
represents a double bond.

13. A compound according to claim 12 wherein one of R^{1′} and R^{2′} are hydrogen and R^{3′} is a carbonylamino, sulfonylamino, carboxyl, or acyl moiety.

14. A compound according to claim 10 wherein R^{1′}, R^{2′}, and R^{3′} are not carboxy, or —SR, and R^{1′}, R^{2′} and R^{3′} are not independently selected.

15. A compound according to claim 10 wherein R^{1′}, R^{2′} and R^{3′} are, independently, carboxy, carbonylamino, sulfonylamino or acyl.

16. A compound according to claim 10 wherein R¹ is hydrogen and R^{2′} and R^{3′} are independently selected from carboxy, carbonylamino, sulfonylamino or acyl.

17. A method for influencing the action of a G-protein-coupled receptor kinase in a cell comprising administering to or contacting with a cell or a mammal in need of treatment at least one compound according to Formula (II), or a method of reducing GPCR desensitization in a cell comprising administering to or contacting the cell or a mammal in need of treatment with a therapeutically effective amount of a compound according to Formula (II), or a method of inhibiting a G-protein-coupled receptor kinase comprising applying or administering to a medium or contacting a cell or a mammal in need of treatment with an effective inhibitory amount of a compound according to Formula (II): 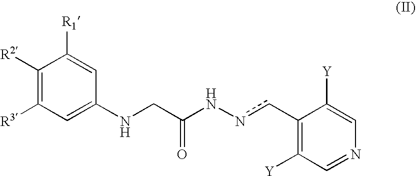
wherein [symbol]
indicates a single or double bond;
R^{1′}, R^{2′} and R^{3′} are independently selected from hydrogen; halogen; C₁-C₄ alkyl; amino; nitro; cyano; heteroaryl; carboxyl, carbonylamino; aminosulfonyl; sulfonylamino; aminoacyl; thioalkyl; sulfonyl; acyl; heterocycle; —OR; —SR, —O—C₁-C₄alkyl-heterocycle; —C(O)NH—C₁-C₄alkyl-heterocycle; —C(O)NH-heteroaryl; —C(O)NH-aryl; or carbonylmethylamino;
R is selected from C₁-C₄ alkyl; aryl, heteroaryl, C₁-C₄ alkyl aryl or C₁-C₄ alkyl heteroaryl; and
Y is selected from hydrogen, halogen or nitrile.

18. A method according to claim 17 wherein Y is H or Cl.

19. A method according to claim 17 wherein [symbol]
represents a double bond.

20. A method according to claim 19 wherein one of R^{1′} and R^{2′} are hydrogen and R^{3′} is a carbonylamino, sulfonylamino, carboxyl, or acyl moiety.

21. A compound according to claim 17 wherein R^{3′} is carbonylamino.

22. A compound according to claim 21 wherein the carbonylamino moiety is selected from C(O)NH-phenyl, C(O)NH-m-pyridyl, C(O)NH-o-pyridyl, C(O)NH-p-pyridyl, C(O)NH₂, or C(O)NHCH₃.

23. The method according to claim 17 wherein the G-protein-coupled receptor kinase is GRK-2, GRK-3, GRK-5 or GRK-6.

24. The method according to claim 23 wherein the G-protein-coupled receptor kinase is GRK-2.

25. A composition comprising:
a) a hydrazide derivative having the structure 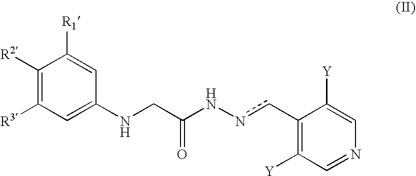
wherein [symbol]
indicates a single or double bond;
R^{1′}, R^{2′} and R^{3′} are independently selected from hydrogen; halogen; C₁-C₄ alkyl; amino; nitro; cyano; heteroaryl; carboxyl, carbonylamino; aminosulfonyl; sulfonylamino; aminoacyl; thioalkyl; sulfonyl; acyl; heterocycle; —OR; —SR, —O—C₁-C₄alkyl-heterocycle; —C(O)NH—C₁-C₄alkyl-heterocycle; —C(O)NH-heteroaryl; —C(O)NH-aryl; or carbonylamino;
R is selected from C₁-C₄ alkyl; aryl, heteroaryl, C₁-C₄ alkyl aryl or C₁-C₄ alkyl heteroaryl;
Y is selected from hydrogen, halogen or nitrile; and
b) a carrier.

26. The composition of claim 25, wherein the carrier is selected from the group consisting of systemic and topical carriers.

27. The composition of claim 25, wherein the composition comprises about 0.01% to 10% of the hydrazide derivative and 90 to 99.99% of the systemic carrier.

28. A method of treating a condition comprising administering to a subject in need of treatment a safe and effective amount of a hydrazide derivative, wherein the condition is selected from the group consisting of eye disease, bone disorder, heart disease, hepatic disease, renal disease, pancreatitis, cancer, myocardial infarct, gastric disturbance, hypertension, fertility control, nasal congestion, neurogenic bladder disorder, a gastrointestinal disorder, and a dermatological disorder.

29. The method of claim 28, wherein the condition comprises eye disease.

30. The method of claim 29, wherein the eye disease comprises glaucoma.

31. The method of claim 30, wherein the hydrazide derivative is of formula 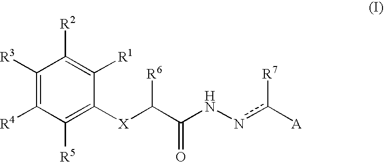
wherein [symbol]
may be a single or double bond;
A is selected from
a heteroaryl group (i): 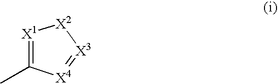
wherein X¹, X², X³ and X⁴ are, independently, CH, O, S or N—R⁶, with the proviso that at least one of X² or X³ is O, S or N—R⁶; and
a heteroaryl group (ii): 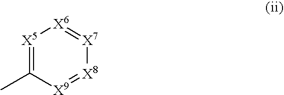
wherein X⁵ and X⁹ are CH or C-halogen, X⁶ and X⁸ are CH, and X⁷ is N, and wherein the six-membered heteroaryl group may be further fused with an unsubstituted six-member aryl group;
R¹, R², R³, R⁴, and R⁵ are, independently, hydrogen; halogen; C₁-C₄ alkyl; amino; nitro; cyano; heteroaryl; carboxy, carbonylamino; aminosulfonyl; sulfonylamino; aminoacyl; thioalkyl; sulfonyl; acyl; heterocycle; —OR; —O—C₁-C₄alkyl-heterocycle; —C(O)NH—C₁-C₄alkyl-heterocycle; —C(O)NH-heteroaryl; —C(O)NH-aryl; or carboxylamino; wherein
R is C₁-C₄ alkyl; aryl, heteroaryl, C₁-C₄ alkyl aryl or C₁-C₄ alkyl heteroaryl;
R⁶ is H or C₁-C₄ alkyl;
R⁷ is hydrogen, C₁-C₄ alkyl or C₁-C₄ alkoxy; and
X is N—R⁶.

32. The method of claim 30, wherein the hydrazide derivative is of formula (II): 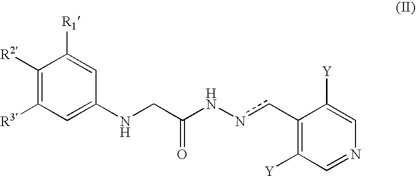
wherein [symbol]
may be a single or double bond;
R^{1′}, R^{2′} and R^{3′} are independently selected from hydrogen; halogen; C₁-C₄ alkyl; amino; nitro; cyano; heteroaryl; carboxyl, carbonylamino; aminosulfonyl; sulfonylamino; aminoacyl; thioalkyl; sulfonyl; acyl; heterocycle; —OR; —SR; —C(O)NH-heteroaryl; or —C(O)NH-aryl;
R is C₁-C₄ alkyl; aryl, heteroaryl, C₁-C₄ alkyl aryl or C₁-C₄ alkyl heteroaryl; and
Y is hydrogen, halogen or nitrile.

33. A compound selected from the following: 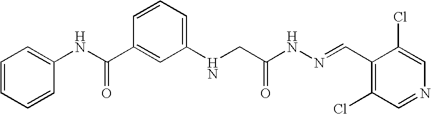 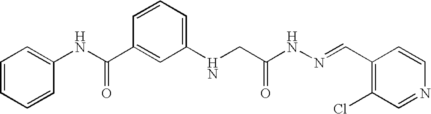 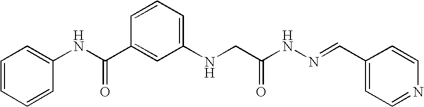 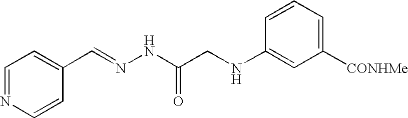 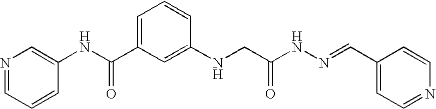 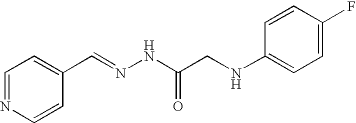 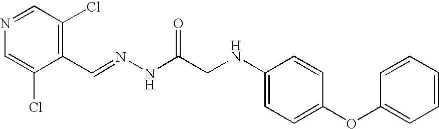 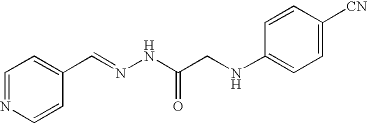 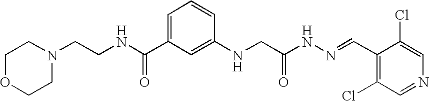 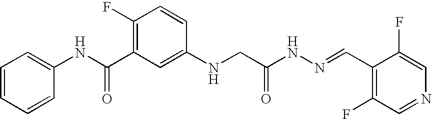 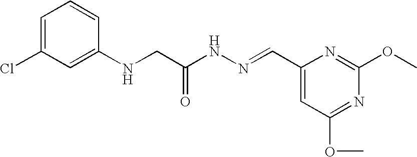 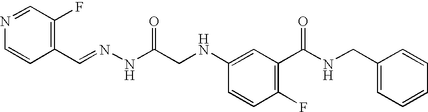 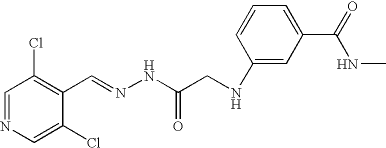 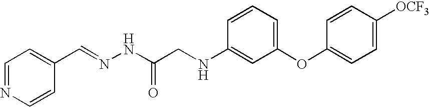 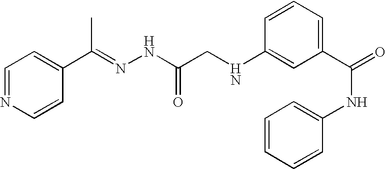 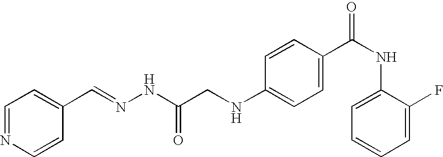 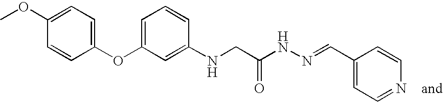 [chemical structure]
